(19) United States
(12) Patent Application Publication
(10) Pub. No.: US 2008/0311047 A1
(43) Pub. Date: Dec. 18, 2008

(54) Multimetric Biosensors and Methods of Using Same

(76) Inventors: Thijs Kaper, Palo Alto, CA (US); Wolf B. Frommer, Stanford, CA (US)

Correspondence Address: MORGAN LEWIS & BOCKIUS LLP, 1111 PENNSYLVANIA AVENUE NW, WASHINGTON, DC, 20004, US

(21) Appl. No.: 12/091,859

(22) PCT Filed: Nov. 16, 2006
(86) PCT No.: PCT/US06/44633

§ 371 Date: Aug. 14, 2008

Related U.S. Application Data
(60) Provisional application No. 60/736,878, filed on Nov. 16, 2005.

(51) Int. Cl. C12N 15/11; C12N 15/12; C12N 15/13; C12N 15/52; C12N 1/21; C12N 1/19; C12N 5/10; A01K 67/027; A01H 5/00; G01N 33/53; A61K 49/00; C12N 15/63

(52) U.S. Cl. 424/9.6; 536/23.1; 536/23.2; 536/23.5; 536/23.53; 435/252.33; 435/254.2; 435/410; 800/13; 435/7.2; 800/298; 435/320.1

(57) ABSTRACT

Multimeric tryptophan biosensors are disclosed, which comprise tryptophan-binding domains conjugated to donor and fluorescent moieties that permit detection and measurement of Fluorescence Resonance Energy Transfer upon tryptophan binding. Such biosensors are useful for real time monitoring of tryptophan metabolism in living cells.

### RELATED APPLICATIONS

This application claims the benefit of priority of U.S. Provisional Application 60/736,878, filed Nov. 16, 2005.

### STATEMENT OF GOVERNMENT SUPPORT

This work was supported by grant No NIH 5 R33 DK 70272. The government may have certain rights to this invention.

### FIELD OF INVENTION

The invention relates generally to the construction of multimeric ligand binding biosensors and methods for measuring and detecting changes in ligand concentration using fluorescence resonance energy transfer (FRET). In particular, the invention provides single chain protein sensors constructed from dimeric proteins such as the tryptophan repressor and other multimeric ligand binding proteins.

### BACKGROUND OF INVENTION

All publications and patent applications herein are incorporated by reference to the same extent as if each individual publication or patent application was specifically and individually indicated to be incorporated by reference.

The publications discussed herein are provided solely for their disclosure prior to the filing date of the present application. Nothing herein is to be construed as an admission that the present invention is not entitled to antedate such publication by virtue of prior invention.

This application is related to International Application PCT/US05/36956, International Application PCT/US05/36957, International Application PCT/US05/36954, International Application PCT/US05/36952, International Application PCT/US05/36957, International Application PCT/US05/36954, and International Application PCT/US05/36953, which are herein incorporated by reference in their entireties.

Tryptophan (Trp or W) is an essential amino acid for mammals, which rely on dietary intake of tryptophan to meet its daily requirements. Tryptophan has a number of interesting medicinal qualities including treatment of insomnia as well as an adjunct in the treatment of a number of psychiatric disorders. Tryptophan levels in human cells depend on transport of tryptophan across the cell membrane. Defects in tryptophan transport in cells or organs may lead to various disorders. For instances, Hartnup disease is an autosomal recessive disorder caused by defective transport of neutral (i.e., monoaminomonocarboxylic) amino acids such as tryptophan in the small intestine and the kidneys.

After absorption, tryptophan circulates in the blood as approximately 80% bound to plasma albumin with the remaining 20% circulating as free tryptophan, and under appropriate conditions, tryptophan is transported into the brain. Once across the blood brain barrier (BBB), tryptophan becomes available for metabolism into serotonin, a neurotransmitter implicated in mood, hunger, and sleep. Low levels of serotonin are associated with depression, fibromyalgia, chronic pain, altered mood, insomnia, PMS, and headaches. Tryptophan metabolism to serotonin also serves well in conditions where depleted serotonin levels exist such as anxiety disorders, obsessive-compulsive disorders, aggression and eating disorders. Parkinson's disease is primarily due to the hypofunction of serotonin nerves, in which serotonin levels are related directly to tryptophan levels.

Subsequently, serotonin, in turn, is metabolized to melatonin, a sleep related hormone produced especially at night in the pineal gland, a small cone-like structure in the epithalamus of the brain that regulates the 24-hour circadian rhythm in humans. Ingestion of a sufficient quantity of tryptophan per se consistently results in reduced sleep latency i.e. the time from “lights out” to sleep, and an improvement in overall quality of sleep through improved sleep architecture (Boman, 1988).

In plants, L-tryptophan is a precursor for auxin, a plant hormone critical for plant growth and that orchestrates many developmental processes. Though many natural and synthetic compounds exhibit auxin-like activity in bioassays, indole-3-acetic acid (IAA) is recognized as the key auxin in most plants. Auxin regulates plant tropic responses (growth toward or away from environmental signals) and apical dominance (repression of branch outgrowth by cells at the shoot tip). Plant growth in response to gravity and light requires asymmetrically distributed auxin across the stem or root. This causes one side to grow more than the other. Similarly, the production of auxin by the “apically dominant” shoot tip, followed by its transport down through the stem, represses the outgrowth of lateral buds.

In bacteria, tryptophan synthesis is regulated by the tryptophan repressor protein (TrpR). TrpR regulates gene expression of the E. coli trpR, trp EDCBA and aroH operons. Purified protein, when activated with L-tryptophan binds to operator DNA sequences (Gunsalus et al., 1980), thus blocking transcription of the structural genes for tryptophan synthesis. The functional unit of TrpR is a dimer in which five of the six helices are interlinked (Schevitz et al. 1985). Two TrpR molecules are necessary to make up two functional binding sites. Binding of L-tryptophan by the TrpR dimer results in conformational changes which promote binding to DNA (Zhang et al. 1987). The L-tryptophan molecule in the TrpR-L-tryptophan complex is directly involved in the interaction with DNA (Otwinowski et al. 1988). TrpR is able to bind a wide variety of tryptophan analogues with varying affinities (Marmorstein et al. 1987). Several of the resulting complexes of TrpR and tryptophan analogues are able to bind to the trp operon sequence (Marmorstein et al. 1989).

Given the important roles tryptophan plays in the normal functioning of plants and living organisms, it would be desirable to provide convenient and real time methods of monitoring tryptophan levels in vitro and in vivo. To be able to measure tryptophan levels directly in living cells, it would be useful to have a nanosensor for tryptophan and its analogs. A tryptophan sensor would be an excellent tool for discovery and drug screening. The response of tryptophan levels could be measured in real time in response to chemicals, metabolic events, transport steps, and signaling processes.

Recently a number of bacterial periplasmic binding proteins (PBP), which undergo a venus flytrap-like closure of two lobes upon substrate binding, have been successfully used as the scaffold of metabolite nanosensors (Fehr et al. 2002; Fehr et al. 2003; Lager et al. 2003). Based on these findings, various fluorescent indicator proteins have been developed for the detection of metabolites such as glucose (Fehr et al. 2004), maltose (Fehr et al. 2002), ribose (Lager et al. 2003) glutamate (Okumoto et al. 2005) (International Application PCT/US05/36956), phosphate (International Application PCT/US05/36955), sucrose (International Application PCT/US05/36951) and polyamine (International Application PCT/US05/36952), each of which is herein incorporated by reference in its entirety.

These sensors consist of a protein of the periplasmic binding protein family, sandwiched between a pair of green fluorescent protein variants fluorescence capable of resonance energy transfer (FRET), the efficiency of which depends on the distance and orientation of the fluorophores. Ligand-binding induced conformational changes in such sensors result in altered FRET signals, which are a measure for the levels of the respective metabolites. The successful development of these biosensors has suggested to the inventors that a tryptophan biosensor may also be constructed because it has been observed that the tryptophan repressor protein also undergoes conformational changes upon binding of L-tryptophan. However, unlike periplasmic binding proteins which are monomers, bacterial tryptophan repressor proteins as discussed above function as dimers.

Recently, FRET has been successfully used to detect formation of multimeric protein complexes. For instance, FRET technology has been used in the detection of multimeric complex formation of estrogen receptor and nuclear coactivators (Liu et al. 2003). FRET has also been applied to the study of homomultimerization of the coxsackievirus 2B protein in living cells by a FRET biosensor comprising one component of the homomultimeric complex such as 2B fused to the fluorescent protein (Van Kuppeveld et al. 2002) and dimerization of mammalian adenylate cyclases by cotransfecting different single unit sensors into the cells (Gu et al. 2002). However, none of these studies has designed and employed single chain biosensors with multimeric or dimeric moieties.

### SUMMARY OF INVENTION

The present inventors have surprisingly found that multimeric biosensors may be successfully constructed by incorporating multiple copies of genes of interest into constructs of the biosensors. Thus, the present invention provides an isolated nucleic acid which encodes a ligand binding fluorescent indicator, the indicator comprising at least one ligand binding protein moiety of a multimeric ligand binding protein complex, a donor fluorophore moiety fused to the ligand binding protein moiety, and an acceptor fluorophore moiety fused to the ligand binding protein moiety.

The present invention further provides tryptophan biosensors that may be used for detecting and measuring changes in tryptophan concentrations in living cells and optimization of the sensors with multimeric tryptophan repressor domains by encoding multiple copies of tryptophan repressor domains in single gene products. Further, the present invention provides use of repressor and/or DNA binding and/or RNA synthesis regulatory proteins for the construction of multimeric ligand binding protein sensors.

In particular, the invention provides an isolated nucleic acid which encodes a tryptophan fluorescent indicator, the indicator comprising at least one tryptophan binding protein moiety of a dimeric tryptophan repressor protein complex, a donor fluorescent protein moiety covalently coupled to the tryptophan binding protein moiety, and an acceptor fluorescent protein moiety covalently coupled to the at least one tryptophan binding protein moiety, wherein fluorescence resonance energy transfer (FRET) between the donor moiety and the acceptor moiety is altered when the donor moiety is excited and tryptophan binds to the tryptophan binding protein moiety. Vectors, including expression vectors, and host cells comprising the inventive nucleic acids are also provided, as well as biosensor proteins encoded by the nucleic acids. Such nucleic acids, vectors, host cells and proteins may be used in methods of detecting tryptophan binding and changes in levels of tryptophan, and in methods of identifying compounds that modulate tryptophan binding or tryptophan-mediated activities.

### BRIEF DESCRIPTION OF THE DRAWINGS

FIGS. 1(A)-1(D) show tryptophan sensors based on the E. coli tryptophan repressor TrpR. (A) TrpR dimer (yellow, red) in complex with L-tryptophan (black) bound to the trp operator (green, blue) (PDB: 1TRO (Otwinowski et al. 1988)). (B) Constructed FLIPW variants. (C) Normalized FRET ratio change of FLIPW-CTY in presence of L-tryptophan (red squares), D-tryptophan (cyan circles), 5-hydroxy-L-tryptophan (yellow squares) and 5-methyl-L-tryptophan (green triangles). (D) Normalized FRET ratio change of FLIPW-CTY (red squares), FLIPW-TCTY (cyan circles), FLIPW-CTYT (green triangles), and FLIPW-CTTY (yellow squares) in the presence of L-tryptophan.

FIG. 2 shows the plasmid map of pTK164, DNA sequence of pTK164 and protein sequence of FLIPW-CTY (SEQ ID NOs: 2 and 3).

FIG. 3 shows the plasmid map of pTK203, DNA sequence of pTK203 and protein sequence of FLIPW-TCTY (SEQ ID NOs: 4 and 5).

FIG. 4 shows the plasmid map of pTK204, DNA sequence of pTK204 and protein sequence of FLIPW-CTYT (SEQ ID NOs: 6 and 7).

FIG. 5 shows the plasmid map of pTK205, DNA sequence of pTK205 and protein sequence of FLIPW-CTTY (SEQ ID NOs: 8 and 9).

FIG. 6 shows the plasmid map of pTK222 and DNA sequence of pTK222 (SEQ ID NO: 10). FLIPW-CTYT as encoded on pTK204 (FIG. 4, SEQ ID NO: 7).

FIGS. 7(A)-7(B) show structural models of FLIPW-CTY and FLIPW-CTYT tryptophan sensors. TrpR: green, magenta (PDB: 1WRP; PDB: Protein data bank at http://www.rcsb.org/pdb/home/home.do), eCFP: blue (based on PDB: 1MYW) and Venus: yellow (PDB: 1MYW). (A) Dimer of two FLIPW-CTY chains resulting in a TrpR dimer that can bind tryptophan. (B) FLIPW-CTYT monomer.

FIGS. 8(A)-8(B) show uptake of tryptophan by COS-7 cell cultures in 96-well microplates monitored with FLIPW-CTYT. (a) FRET ratio change of cell cultures in presence of Tyrode's buffer (squares) and 100 μM L-Trp in Tyrode's buffer (circles). Data correspond to means±S.E. (n=12). (b) Velocity of intracellular FLIPW-CTYT response versus external tryptophan concentration fitted with the Michaelis-Menten equation. Cells were incubated with 0.05, 0.1, 0.25, 0.5, 1, 5, 10 and, 25 μM L-Trp. Data correspond to means±S.E. (n=6).

FIGS. 9(A)-9(B) show hypoxanthine sensor based on the corepressor-binding domain of E. coli PurR. (a) PurR dimer (red, yellow) in complex with hypoxanthine (black) bound to the purF operator site (blue) (PDB: 1PNR (22)). (b) Saturation of the FLIPpur sensor in the presence of hypoxanthine.

FIG. 10 shows relative position of the components of the FLIPW-CTYT sensor. The TrpR dimer (green, magenta, PDB: 1WRP) and Venus (yellow, PDB: 1MYW) are modeled to be sterically compatible, with the termini approaching within 1 Å.

FIGS. 11(A)-11(B) show structural models of FLIPW-TCTY and FLIPW-CTYT tryptophan sensors. TrpR: green, magenta (PDB: 1WRP), eCFP: blue (based on PDB: 1MYW) and Venus: yellow (PDB: 1MYW). (a) FLIPW-TCTY monomer, (b) FLIPW-CTYT monomer.

FIG. 12 shows perfusion of HEK293T cells transfected with pTK222. Cells were perfused with Tyrode's buffer. Between 1′30″ and 3′ (indicated by triangles) buffer was supplemented with 10 μM L-tryptophan. Response of the sensor is determined from the ratio of fluorescence output at 528 nm and 485 nm. During perfusion with tryptophan, the intracellular tryptophan levels increase. The Trp levels decrease during subsequent perfusion with buffer due to efflux and metabolism.

### DETAILED DESCRIPTION OF THE INVENTION

The following description includes information that may be useful in understanding the present invention. It is not an admission that any of the information provided herein is prior art or relevant to the presently claimed inventions, or that any publication specifically or implicitly referenced is prior art.

Other objects, advantages and features of the present invention become apparent to one skilled in the art upon reviewing the specification and the drawings provided herein. Thus, further objects and advantages of the present invention will be clear from the description that follows.

Biosensors

The present invention provides biosensors of multimeric ligand binding proteins for detecting and measuring changes in analyte concentrations using Fluorescence Resonance Energy Transfer (FRET). One embodiment, among others, is an isolated nucleic acid which encodes a ligand binding fluorescent indicator, the indicator comprising: at least one ligand binding protein moiety of a multimeric ligand binding protein complex, a donor fluorescent protein moiety covalently coupled to the ligand binding protein moiety, and an acceptor fluorescent protein moiety covalently coupled to the ligand binding protein moiety, wherein FRET between the donor moiety and the acceptor moiety is altered when the donor moiety is excited and ligand binds to the ligand binding protein moiety.

As used herein, the term “multimer” and grammatical variations thereof refer to formation of a multimeric complex between two or more distinct molecules. The multimer complex may comprise, for example, two or more molecules of the same protein (e.g., a homo-dimer, -trimer, -tetramer or higher order multimer) or a mixture of two or more different (i.e., non-identical) proteins (e.g. a hetero-dimer, -trimer, -tetramer or higher multimer). For example, multimeric antibodies may comprise the same antibody or two or more different antibodies, each of which have two or more functions or activities (e.g., bind to two or more epitopes).

As used herein, “covalently coupled” means that the donor and acceptor fluorescent moieties may be conjugated to the ligand binding protein moiety via a chemical linkage, for instance to a selected amino acid in said ligand binding protein moiety. Covalently coupled also means that the donor and acceptor moieties may be genetically fused to the ligand binding protein moiety such that the ligand binding protein moiety is expressed as a fusion protein comprising the donor and acceptor moieties.

The isolated nucleic acid that encodes the multimeric ligand binding protein moiety can be any nucleic acid, and preferably is the nucleic acid that encodes portions of multimeric proteins. In one embodiment, the isolated nucleic acid of interest encodes a hetero- or homo-dimer, -trimer, -tetramer, -pentamer, -hexamer or higher order multimer. Multimeric proteins may be selected, for example, from a binding protein (e.g. an antigen binding polypeptide), enzyme, receptor, ligand, nucleic acid binding protein (e.g. a repressor protein binding DNA), growth regulatory factor, differentiative factor, and chemotactic factor. For example, the repressor protein, lac repressor acts as a tetramer and the tyrosine repressor acts as a hexamer.

Nucleic acids encoding protein and peptide hormones are a preferred class of nucleic acids of interest in the present invention. Such protein and peptide hormones are synthesized throughout the endocrine system and include, but are not limited to, hypothalamic hormones and hypophysiotropic hormones, anterior, intermediate and posterior pituitary hormones, pancreatic islet hormones, hormones made in the gastrointestinal system, renal hormones, thymic hormones, parathyroid hormones, adrenal cortical and medullary hormones. Specifically, hormones that can be utilized by the present invention include, but are not limited to, chorionic gonadotropin, corticotropin, erythropoietin, glucagons, IGF-1, oxytocin, platelet-derived growth factor, vascular endothelial growth factor, calcitonin, follicle-stimulating hormone, luteinizing hormone, thyroid-stimulating hormone, insulin, gonadotropin-releasing hormone and its analogs, vasopressin, octreotide, somatostatin, prolactin, adrenocorticotropic hormone, antidiuretic hormone, thyrotropin-releasing hormone (TRH), growth hormone-releasing hormone (GHRH), dopamine, melatonin, thyroxin (T₄), parathyroid hormone (PTH), glucocorticoids such as cortisol, mineralocorticoids such as aldosterone, androgens such as testosterone, adrenaline (epinephrine), noradrenaline (norepineplirine), estrogens such as estradiol, progesterone, glucagons, calcitrol, calciferol, atrial-natriuretic peptide, gastrin, secretin, cholecystokinin (CCK), neuropeptide Y, ghrelin, PYY₃₋₃₆, angiotensinogen, thrombopoietin, and leptin.

Further included in the present invention are nucleic acids of interest that encode multimeric receptors. Multimeric receptors include homodimers (e.g., PDGF receptor αα, and ββ isoforms, erythropoietin receptor, MPL, and G-CSF receptor), heterodimers whose subunits each have ligand-binding and effector domains (e.g., PDGF receptor αβ isoform), and multimers having component subunits with disparate functions (e.g., IL-2, IL-3, IL-4, IL-5, IL-6, IL-7, and GM-CSF receptors). Some receptor subunits are common to a plurality of receptors. For example, the AIC2B subunit, which cannot bind ligand on its own but includes an intracellular signal transduction domain, is a component of IL-3 and GM-CSF receptors. Many cytokine receptors can be placed into one of four related families on the basis of the structure and function. Hematopoietic receptors, for example, are characterized by the presence of a domain containing conserved cysteine residues and the WSXWS motif. Cytokine receptor structure has been reviewed by Urdal, Ann. Reports Med. Chem. 26:221-228, 1991 and Cosman, Cytokine 5:95-106, 1993. Under selective pressure for organisms to acquire new biological functions, new receptor family members likely arise from duplication of existing receptor genes leading to the existence of multi-gene families. Family members thus contain vestiges of the ancestral gene, and these characteristic features can be exploited in the isolation and identification of additional family members. Thus, the cytokine receptor superfamily is subdivided into several families, for example, the immunoglobulin family (including CSF-1, MGF, IL-1, and PDGF receptors); the hematopoietin family (including IL-2 receptor β-subunit, GM-CSF receptor α-subunit, GM-CSF receptor β-subunit; and G-CSF, EPO, IL-3, IL-4, IL-5, IL-6, IL-7, and IL-9 receptors); TNF receptor family (including TNF (p80) TNF (p60) receptors, CD27, CD30, CD40, Fas, and NGF receptor). Multimeric receptors also include hormone receptors (TSH, FSH, CG, VEGF, PDGF, EGF, etc.), steroid receptors, serotonin receptors, dopamine receptors, metabotropic and ionotropic glutamate receptors, insulin receptors, IGF1 receptors, G-protein-coupled receptors (including leukotriene B(4) receptor and BLT1 as dimer).

Other multimeric proteins that may be utilized using the present invention are as follows: factors involved in the synthesis or replication of DNA, such as DNA polymerase alpha and DNA polymerase delta; proteins involved in the production of mRNA, such as TFIID and TFIIH; cell, nuclear and other membrane-associated proteins, such as hormone and other signal transduction receptors, active transport proteins and ion channels, multimeric proteins in the blood, including hemoglobin, fibrinogen and von Willabrand's Factor; proteins that form structures within the cell, such as actin, myosin, and tubulin and other cytoskeletal proteins; proteins that form structures in the extra cellular environment, such as collagen, elastin and fibronectin; proteins involved in intra- and extra-cellular transport, such as kinesin and dynein, the SNARE family of proteins (soluble NSF attachment protein receptor) and clathrin; proteins that help regulate chromatin structure, such as histones and protamines, Swi3p, Rsc8p and moira; multimeric transcription factors such as Fos, Jun and CBTF (CCAAT box transcription factor); multimeric enzymes such as acetylcholinesterase and alcohol dehydrogenase; chaperone proteins such as GroE, Gro EL (chaperonin 60) and Gro ES (chaperonin 10); anti-toxins, such as snake venom, botulism toxin, Streptococcus super antigens; lysins (enzymes from bacteriophage and viruses); as well as most allosteric proteins.

In another embodiment, the present invention provides an isolated nucleic acid which encodes a ligand binding fluorescent indicator, the indicator comprising: at least one ligand binding protein from a repressor protein, a donor fluorescent protein moiety covalently coupled to the ligand binding protein moiety, and an acceptor fluorescent protein moiety covalently coupled to the ligand binding protein moiety, wherein FRET between the donor moiety and the acceptor moiety is altered when the donor moiety is excited and ligand binds to the ligand binding protein moiety. As used herein, the term “repression” refers to transcriptional repression as by a transcriptional repressor such as a DNA binding transcriptional repressor, which binds a target promoter to be repressed.

Suitable repressor proteins may also include, but are not limited to, lactose, galactose, purine, tetracycline, tyrosine, tryptophan repressor proteins, multidrug-binding protein QacR, arabinose (AraC), mercury (MerR), histone deacetylase (HDAC), MEF2-interacting transcription repressor (MITR), silencing mediator for retinoid and thyroid hormone receptors (SMRT), nuclear corepressor (N-CoR), Small Unique Nuclear receptor CoRepressor (SUN-CoR), TG interacting factor (TGIF), Sloan Kettering virus oncogene homolog (Ski), Ski-related novel gene (Sno), NGFI-A-binding protein (NAB), or Friend of GATA (FOG).

In yet another embodiment, the invention provides isolated nucleic acids encoding tryptophan binding fluorescent indicators and the tryptophan fluorescent indicators encoded thereby. The embodiment, among others, is an isolated nucleic acid which encodes a tryptophan binding fluorescent indicator, the indicator comprising: at least one tryptophan binding protein moiety of a multimeric ligand binding protein complex, a donor fluorescent protein moiety covalently coupled to the tryptophan binding protein moiety, and an acceptor fluorescent protein moiety covalently coupled to the tryptophan binding protein moiety, wherein FRET between the donor moiety and the acceptor moiety is altered when the donor moiety is excited and tryptophan binds to the tryptophan binding protein moiety.

As an example, the tryptophan binding protein moiety, among others, is a tryptophan binding protein moiety from E. coli having the following sequence:

### MAQQSPYSAA MAEQRHQEWL RFVDLLKNAY QNDLHLPLLN LMLTPDEREA LGTRVRIVEE LLRGEMSQRE LKNELGAGIA TITRGSNSLK AAPVELRQWL EEVLLKSD (SEQ ID NO: 1).

Any portion of the tryptophan repressor DNA sequence which encodes a tryptophan binding region may be used in the nucleic acids of the present invention. Tryptophan binding portions of tryptophan binding protein (BP) or any of its homologues from other organisms, for instance Gram negative bacteria including thermophilic and hyperthermophilic organisms, may be cloned into the vectors described herein and screened for activity according to the disclosed assays. Ligand binding proteins of thermophilic and hyperthermophilic organisms are particularly useful for constructing sensors having increased stability and resistance to heat or harsh environmental conditions. See International Application PCT/US05/36954, which is herein incorporated by reference in its entirety.

Naturally occurring species variants of tryptophan BP may also be used, in addition to artificially engineered variants comprising site-specific mutations, deletions or insertions that maintain measurable tryptophan binding function. Variant nucleic acid sequences suitable for use in the nucleic acid constructs of the present invention will preferably have at least 30, 40, 50, 60, 70, 75, 80, 85, 90, 95, or 99% similarity or identity to the gene sequence for tryptophan BP. Suitable variant nucleic acid sequences may also hybridize to the gene for tryptophan BP under highly stringent hybridization conditions. High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993), which is herein incorporated by reference. Generally, stringent conditions are selected to be about 5-10° C. lower than the thermal melting point (Tm) for the specific sequence at a defined ionic strength pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide.

Preferred artificial variants of the present invention may be designed to exhibit decreased affinity for the ligand, in order to expand the range of ligand concentration that can be measured by the disclosed nanosensors. Additional artificial variants showing decreased or increased binding affinity for ligands may be constructed by random or site-directed mutagenesis and other known mutagenesis techniques, and cloned into the vectors described herein and screened for activity according to the disclosed assays. The binding specificity of disclosed biosensors may also be altered by mutagenesis so as to alter the ligand recognized by the biosensor. See, for instance, Looger et al., Nature, 423 (6936): 185-190.

The sensors of the invention may also be designed with tryptophan binding moieties and one or more additional protein binding moieties that are covalently coupled or fused together and to the donor and acceptor fluorescent moieties in order to generate an allosteric enzyme whose activity is controlled by more than one ligand. Allosteric enzymes containing dual specificity for more than one ligand have been described in the art, and may be used to construct the FRET biosensors described herein (Guntas and Ostermeier, 2004, J. Mol. Biol. 336(1): 263-73).

As described herein, the donor and acceptor moieties may be fused to the termini of the at least one ligand binding moiety of a multimeric ligand binding protein complex or to an internal position within the at least one ligand binding moiety of a multimeric ligand binding protein complex so long as FRET between the donor moiety and the acceptor moiety is altered when the donor moiety is excited and a ligand binds to the ligand binding protein moiety. See International Application PCT/US05/36957, which is herein incorporated by reference in its entirety.

The isolated nucleic acids of multimeric binding protein complex of the invention may comprise a structure according to the following formula (I):

A-B-C-D (I)

wherein A and C are fluorophore moieties, and B and D are ligand binding protein moieties.

In another embodiment, the present invention provides an isolated nucleic acid, wherein said ligand binding fluorescent indicator comprises a structure of formula (I), wherein A and C are ligand binding protein moieties, and B and D are fluorophore moieties.

In yet another embodiment, the present invention provides an isolated nucleic acid, wherein said ligand binding fluorescent indicator comprises a structure of formula (I), wherein A and D are ligand binding protein moieties, and B and C are fluorophore moieties.

In yet another embodiment, the present invention provides an isolated nucleic acid, wherein said ligand binding fluorescent indicator comprises a structure of formula (I), wherein A and D are fluorophore moieties, and B and C are ligand binding protein moieties.

The ligand binding protein moieties may be from separate proteins of a multimeric ligand binding protein complex. Thus, the present invention provides an isolated nucleic acid with two or more polynucleotide moieties, each of which encodes a ligand binding protein that forms a part of the multimeric protein complex wherein the nucleic acid encodes a protein comprising a donor fluorophore moiety fused to the two or more ligand binding protein moieties, and an acceptor fluorophore moiety fused to the two or more ligand binding protein moieties.

The isolated nucleic acids of the invention may incorporate any suitable donor and acceptor fluorescent protein moieties that are capable in combination of serving as donor and acceptor moieties in FRET. Preferred donor and acceptor moieties are selected from the group consisting of GFP (green fluorescent protein), CFP (cyan fluorescent protein), BFP (blue fluorescent protein), YFP (yellow fluorescent protein), and enhanced variants thereof such as enhanced YFP (EYFP), with a particularly preferred embodiment provided by the donor/acceptor pair CFP/YFP Venus, a variant of YFP with improved pH tolerance and maturation time (Nagai et al. 2002). A variant of yellow fluorescent protein with fast and efficient maturation for cell-biological applications. Nat. Biotechnol. 20, 87-90). An alternative is the MiCy/mKO pair with higher pH stability and a larger spectral separation (Karasawa et al. 2004). Also suitable as either a donor or acceptor is native DsRed from a Discosoma species, an ortholog of DsRed from another genus, or a variant of a native DsRed with optimized properties (e.g. a K83M variant or DsRed2 (available from Clontech)). Criteria to consider when selecting donor and acceptor fluorescent moieties is known in the art, for instance as disclosed in U.S. Pat. No. 6,197,928, which is herein incorporated by reference in its entirety.

As used herein, the term “variant” is intended to refer to polypeptides with at least about 30%, 40%, 50%, 60%, 70%, more preferably at least 75% identity, including at least 80%, 90%, 95% or greater identity to native fluorescent molecules. Many such variants are known in the art, or can be readily prepared by random or directed mutagenesis of a native fluorescent molecules (see, for example, Fradkov et al., FEBS Lett. 479:127-130 (2000)).

When the fluorophores of the biosensor contain stretches of similar or related sequence(s), the present inventors have recently discovered that gene silencing may adversely affect expression of the biosensor in certain cells and particularly whole organisms. In such instances, it is possible to modify the fluorophore coding sequences at one or more degenerate or wobble positions of the codons of each fluorophore, such that the nucleic acid sequences of the fluorophores are modified but not the encoded amino acid sequences. Alternatively, one or more conservative substitutions that do not adversely affect the function of the fluorophores may also be incorporated. See PCT application [Attorney Docket No. 056100-5054, “Methods of Reducing Repeat-Induced Silencing of Transgene Expression and Improved Fluorescent Biosensors], which is herein incorporated by reference in its entirety.

It is also possible to use or luminescent quantum dots (QD) for FRET (Clapp et al., 2005, J. Am. Chem. Soc. 127(4): 1242-50), dyes, including but not limited to TOTO dyes (Laib and Seeger, 2004, J Fluoresc. 14(2):187-91), Cy3 and Cy5 (Churchman et al., 2005, Proc Natl Acad Sci USA. 102(5): 1419-23), Texas Red, fluorescein, and tetramethylrhodamine (TAMRA) (Unruh et al., Photochem Photobiol. 2004 Oct. 1), AlexaFluor 488, to name a few, as well as fluorescent tags (see, for example, Hoffman et al., 2005, Nat. Methods 2(3): 171-76).

The invention further provides vectors containing isolated nucleic acid molecules encoding the biosensor polypeptides described herein. Exemplary vectors include vectors derived from a virus, such as a bacteriophage, a baculovirus or a retrovirus, and vectors derived from bacteria or a combination of bacterial sequences and sequences from other organisms, such as a cosmid or a plasmid. Such vectors include expression vectors containing expression control sequences operatively linked to the nucleic acid sequence coding for the biosensor. Vectors may be adapted for function in a prokaryotic cell, such as E. coli or other bacteria, or a eukaryotic cell, including animal cells or plant cells. For instance, the vectors of the invention will generally contain elements such as an origin of replication compatible with the intended host cells, one or more selectable markers compatible with the intended host cells and one or more multiple cloning sites. The choice of particular elements to include in a vector will depend on factors such as the intended host cells, the insert size, whether regulated expression of the inserted sequence is desired, i.e., for instance through the use of an inducible or regulatable promoter, the desired copy number of the vector, the desired selection system, and the like. The factors involved in ensuring compatibility between a host cell and a vector for different applications are well known in the art.

Preferred vectors for use in the present invention will permit cloning of the tryptophan binding domain or receptor between nucleic acids encoding donor and acceptor fluorescent molecules, resulting in expression of a chimeric or fusion protein comprising the tryptophan binding domain covalently coupled to donor and acceptor fluorescent molecules. Exemplary vectors include the bacterial pFLIP derivatives disclosed in Fehr et al. (2002) (Visualization of maltose uptake in living yeast cells by fluorescent nanosensors, Proc. Natl. Acad. Sci. USA 99, 9846-9851), which is herein incorporated by reference in its entirety. Methods of cloning nucleic acids into vectors in the correct frame so as to express a fusion protein are well known in the art.

The tryptophan biosensors of the present invention may be expressed in any location in the cell, including the cytoplasm, cell surface or subcellular organelles such as the nucleus, vesicles, ER, vacuole, etc. Methods and vector components for targeting the expression of proteins to different cellular compartments are well known in the art, with the choice dependent on the particular cell or organism in which the biosensor is expressed. See, for instance, Okumoto, S., Looger, L. L., Micheva, K. D., Reimer, R. J., Smith, S. J., and Frommer, W. B. (2005) P Natl Acad Sci USA 102(24), 8740-8745; Fehr, M., Lalonde, S., Ehrhardt, D. W., and Frommer, W. B. (2004) J Fluoresc 14(5), 603-609, which are herein incorporated by reference in their entireties.

The chimeric nucleic acids of the present invention may be constructed such that the donor and acceptor fluorescent moiety coding sequences are fused to separate termini of the ligand binding domain in a manner such that changes in FRET between donor and acceptor may be detected upon ligand binding. Fluorescent domains can optionally be separated from the ligand binding domain by one or more flexible linker sequences. Such linker moieties are preferably between about 1 and 50 amino acid residues in length, and more preferably between about 1 and 30 amino acid residues. Linker moieties and their applications are well known in the art and described, for example, in U.S. Pat. Nos. 5,998,204 and 5,981,200, and Newton et al., Biochemistry 35:545-553 (1996). Alternatively, shortened versions of linkers or any of the fluorophores described herein may be used. For example, the inventors have shown that deleting N- or C-terminal portions of any of the three modules can lead to increased FRET ratio changes, as described in Application Ser. No. 60/658,141, which is herein incorporated by reference in its entirety.

It will also be possible depending on the nature and size of the ligand binding domains to insert one or both of the fluorescent molecule coding sequences within the open reading frames of the binding proteins such that the fluorescent moieties are expressed and displayed from a location within the biosensor rather than at the termini. Such sensors are generally described in U.S. application Ser. No. 60/658,141, which is herein incorporated by reference in its entirety. It will also be possible to insert a ligand binding sequence into a single fluorophore coding sequence, i.e. a sequence encoding a GFP, YFP, CFP, BFP, etc., rather than between tandem molecules. According to the disclosures of U.S. Pat. No. 6,469,154 and U.S. Pat. No. 6,783,958, each of which is incorporated herein by reference in their entirety, such sensors respond by producing detectable changes within the protein that influence the activity of the fluorophore.

The invention also includes host cells transfected with a vector or an expression vector of the invention, including prokaryotic cells, such as E. coli or other bacteria, or eukaryotic cells, such as yeast cells, animal cells or plant cells. In another aspect, the invention features a transgenic non-human animal having a phenotype characterized by expression of the nucleic acid sequence coding for the expression of the environmentally stable biosensor. The phenotype is conferred by a transgene contained in the somatic and germ cells of the animal, which may be produced by (a) introducing a transgene into a zygote of an animal, the transgene comprising a DNA construct encoding the tryptophan biosensor; (b) transplanting the zygote into a pseudopregnant animal; (c) allowing the zygote to develop to term; and (d) identifying at least one transgenic offspring containing the transgene. The step of introducing of the transgene into the embryo can be achieved by introducing an embryonic stem cell containing the transgene into the embryo, or infecting the embryo with a retrovirus containing the transgene. Transgenic animals of the invention include transgenic C. elegans and transgenic mice and other animals. Transgenic plants are also included.

The present invention also encompasses isolated biosensor molecules having the properties described herein, particularly tryptophan binding fluorescent indicators. Such polypeptides may be recombinantly expressed using the nucleic acid constructs described herein, or produced by chemically coupling some or all of the component domains. The expressed polypeptides can optionally be produced in and/or isolated from a transcription-translation system or from a recombinant cell, by biochemical and/or immunological purification methods known in the art. The polypeptides of the invention can be introduced into a lipid bilayer, such as a cellular membrane extract, or an artificial lipid bilayer (e.g. a liposome vesicle) or nanoparticle.

Methods of Detecting Ligands

In one aspect, the present invention provides methods for the rapid and efficient detection of a plurality of ligand samples using a biosensor of multimeric ligand binding moieties. The methods of the invention can be utilized with any ligand. The ligand may be monovalent, divalent or polyvalent. Exemplary ligands that can be used in the methods of the invention include, but are not limited to, proteins, including, but not limited to, antibodies (or fragments thereof), receptors and enzymes; nucleic acids; carbohydrates; lipids; and small molecules. Similarly, the methods of the invention can be used with any binding partner. As used herein, a binding partner is a molecule that binds to one, two or more multimeric ligand binding moieties of the biosensor in a specific manner. The binding partner can be monovalent, bivalent or polyvalent. Exemplary binding partners that can be used in the methods of the invention include, but are not limited to, proteins, including, but not limited to, antigens, polyclonal antibodies, monoclonal antibodies, single chain antibodies, (scFv), F(ab) fragments, F(ab′)₂ fragments, Fv fragments, receptors and enzymes; nucleic acids; oligonucleotides, carbohydrates such as monosaccharides, disaccharides, polysaccharides; lipids, fatty acids, amino acids, oligopeptides, polypeptides, proteoglycans, glycoprotein, natural or synthetic polymers, and small molecular weight compounds such as drugs or drug candidates, cell, virus, bacteria, and biological sample. A biological sample can be, for example, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, and prostatitc fluid.

In a preferred embodiment, the ligand or binding partner is tryptophan. Thus, the biosensor nucleic acids and proteins of the present invention are useful for detecting tryptophan binding and measuring changes in the levels of tryptophan both in vitro and in a plant or an animal. In one embodiment, the invention comprises a method of detecting changes in the level of tryptophan in a sample of cells, comprising (a) providing a cell expressing a nucleic acid encoding a tryptophan biosensor as described herein and a sample of cells; and (b) detecting a change in FRET between a donor fluorescent protein moiety and an acceptor fluorescent protein moiety, each covalently attached to the tryptophan binding domain, wherein a change in FRET between said donor moiety and said acceptor moiety indicates a change in the level of tryptophan in the sample of cells.

FRET may be measured using a variety of techniques known in the art. For instance, the step of determining FRET may comprise measuring light emitted from the acceptor fluorescent protein moiety. Alternatively, the step of determining FRET may comprise measuring light emitted from the donor fluorescent protein moiety, measuring light emitted from the acceptor fluorescent protein moiety, and calculating a ratio of the light emitted from the donor fluorescent protein moiety and the light emitted from the acceptor fluorescent protein moiety. The step of determining FRET may also comprise measuring the excited state lifetime of the donor moiety or anisotropy changes (Squire A, Verveer P J, Rocks O, Bastiaens P I. J Struct Biol. 2004 July; 147(1):62-9. Red-edge anisotropy microscopy enables dynamic imaging of homo-FRET between green fluorescent proteins in cells.). Such methods are known in the art and described generally in U.S. Pat. No. 6,197,928, which is herein incorporated by reference in its entirety.

The amount of tryptophan and its analogs in a sample of cells can be determined by determining the degree of FRET. First the sensor must be introduced into the sample. Changes in tryptophan concentration can be determined by monitoring FRET at a first and second time after contact between the sample and the fluorescent indicator and determining the difference in the degree of FRET. The amount of tryptophan in the sample can be quantified for example by using a calibration curve established by titration.

The cell sample to be analyzed by the methods of the invention may be contained in vivo, for instance in the measurement of tryptophan transport or signaling on the surface of cells, or in vitro, wherein tryptophan efflux may be measured in cell culture. Alternatively, a fluid extract from cells or tissues may be used as a sample from which tryptophan is detected or measured.

Methods for detecting tryptophan levels as disclosed herein may be used to screen and identify compounds that may be used to modulate tryptophan concentrations and activities relating to tryptophan changes. In one embodiment, among others, the invention comprises a method of identifying a compound that modulates tryptophan homeostasis (metabolism & uptake) or levels comprising (a) contacting a mixture comprising a cell expressing a tryptophan biosensor as disclosed herein and a sample of cells with one or more test compounds, and (b) determining FRET between said donor fluorescent domain and said acceptor fluorescent domain following said contacting, wherein increased or decreased FRET following said contacting indicates that said test compound is a compound that modulates tryptophan binding activity or tryptophan levels.

The term “modulate” in this embodiment means that such compounds may increase or decrease tryptophan binding homeostasis (metabolism & uptake) activity, or may affect activities, i.e., cell functions or signaling cascades, that affect tryptophan levels. Compounds that increase or decrease tryptophan homeostasis (metabolism & uptake) activity may be targets for therapeutic intervention and treatment of disorders associated with aberrant tryptophan activity, or with aberrant cell metabolism or signal transduction, as described above. Other compounds that increase or decrease tryptophan homeostasis (metabolism & uptake) activity or tryptophan levels associated with cellular functions may be developed into therapeutic products for the treatment of disorders associated with ligand binding activity.

Utilities

The multimeric sensors of the present invention will be useful for a wide range of applications. The sensors may be expressed in living plant and animal cells where they may be used for monitoring steady state levels of ligands, both in vivo and in vitro. In particular, when the tryptophan biosensors are expressed in bacterial or yeast cells they can be used for screening of genome libraries for the identification and cloning of tryptophan transporters. When expressed in mammalian cells, the sensors can be used in screens for the identification of drugs that influence the uptake of tryptophan. Increased uptake of tryptophan will diminish the symptoms associated with a variety of medical conditions such as Hartnup disease and depression. Compounds that lead to a decrease in tryptophan levels might serve as drug leads for treatment of Parkinson's disease. Thus, the multimeric tryptophan sensors can be used to monitor progress of tryptophan treatment in various diseases. Alternatively, the biosensor can be used to screen and identify compounds that may be used to modulate tryptophan concentrations and activities.

The tryptophan sensors may also serve as a basis for the development of sensors for the detection of tryptophan derivatives such as indoles, serotonin, and melatonin. Serotonin sensors can be used for identification of drugs for the treatment of above-mentioned medical conditions by analysis of the effect of chemical libraries on serotonin levels in the neuron cells and synaptic cleft. Melatonin sensors expressed in pinal gland cells can be used for identification of compounds that effect melatonin production levels, which would provide drug leads for treatment of insomnia.

The tryptophan sensors may also serve as a useful tool in measuring tryptophan levels in plant. Tryptophan sensors expressed in plant cells allow for identification of agents that can up regulate or down regulate tryptophan biosynthesis and that can be used as additions to fertilizers. Tryptophan levels in plant could be important for auxin levels as tryptophan is a precursor of auxin, of which indole-3-acetic acid (IAA) is the most prominent. Sensors for tryptophan thus serve as basis for development of sensors for auxin which both have potential for several applications that can lead to development of improved crops or be used as tools for plant metabolite analysis. Plant development is under tight control of temporal and spatial auxin levels. IAA sensors can be used for identification of these levels and locations of auxin action. Because auxin is transported throughout the plant, auxin sensors expressed in yeast cells can be used for identification of auxin transporters by screening of genomic plant DNA libraries. Auxin levels are also indicative for crop development. Thus, auxin sensors can be used for rapid and easy analysis of auxin levels in the field and thus aid in determination of optimal harvest yields.

The present invention provides a method of determining the amount of a ligand, such as Trp. As an example, the method of the present invention comprises contacting a biological sample with a ligand binding fluorescent indicator containing a ligand binding protein moiety of a multimeric ligand binding protein complex, a donor fluorophore fused to the ligand binding protein moiety, and an acceptor fluorophore fused to the ligand binding protein moiety; and monitoring the level of FRET in the biological sample as a measure of the level of ligand, such as Trp in the sample.

The present invention also provides a method of diagnosing diseases associated with abnormal amounts of a ligand in a subject. The present invention provides methods of monitoring the onset, progression or regression of a disease associated with a ligand by detecting the level of a ligand in a subject. As an example, detecting the amount of Trp in a biological sample by monitoring the level of FRET in the biological sample containing the ligand binding fluorescent indicator of the present invention.

In one embodiment, the present invention provides methods of using the ligand binding fluorescent indicator of the present invention to identify agonists and antagonists that modulate the binding of a ligand, such as Trp. As an example, the present invention may be used to identify agonists and antagonists of Trp metabolism. In another embodiment the present invention provides methods of using the ligand binding fluorescent indicator of the present invention to evaluate the effect of a pharmacological agent on ligand binding, such as the binding of Trp to TrpR.

As described of above, the methods of the present invention comprises monitoring the level of FRET. Monitoring FRET in a sample containing the ligand binding fluorescent indicator may involve measuring FRET, detecting FRET or detecting a FRET signal by methods known to a person of ordinary skill in the art. Monitoring FRET may also involve comparing FRET measured from control samples.

In one aspect, the ligand binding fluorescent indicators of the present invention are used with biological samples. The biological samples may comprise cells, tissues, or bodily fluid from a subject such as an animal or a plant. In another aspect, the methods of the present invention are applicable to the in vivo use of the ligand binding fluorescent indicator in a subject and the analysis of ligands such as metabolites and nutrients in vivo.

The present invention also provides kits for various uses of the ligand binding fluorescent indicator such as determining the amount of Trp in a sample or diagnosing a disease associated with abnormal amounts of Trp. The kits include a ligand binding fluorescent indicator of the present invention and instructions for the use of the indicator. The kit may also provide a method for measuring or detecting FRET.

Tryptophan Sensor

L-Tryptophan is an essential amino acid and is necessary for protein synthesis in mammalian cells. In addition, it is the precursor for the inhibitory neurotransmitter serotonin, the circadian-clock-regulating hormone melatonin, and vitamin B3 niacin, necessary for the synthesis of coenzymes NAD and NADP. Degradation products of L-tryptophan are involved in suppression of T-cell mediated immune response. Mammalian cells cannot synthesize L-tryptophan and depend on transport machineries for its uptake. Traditionally, uptake has been determined using radiolabeled substrates, and levels have been measured in cell extracts via LC/GC-MS. Both methods are neither time-resolved nor specific, and lack high temporal or cellular/subcellular resolution. Given the importance of L-tryptophan for human health, an analytical tool for non-invasive, time-resolved determination of intracellular L-tryptophan levels was deemed highly desirable.

Fluorescent indicator proteins (FLIPs) have been successful tools for real-time monitoring of metabolite levels in living cells. Typically, the nanosensors consist of a ligand-sensing domain, allosterically coupled to a pair of green fluorescent protein variants capable of resonance energy transfer (FRET), the efficiency of which depends on the distance between and relative orientation of the fluorophore dipoles. Ligand-binding induced conformational changes in the sensors result in altered FRET efficiencies, which correlate with the levels of the respective metabolites. Periplasmic binding proteins (PBPs) have been successfully exploited for the construction of FLIPs for imaging of key metabolites such as glucose (Fehr et al. 2003), maltose (Fehr et al. 2002), ribose (Lager et al. 2003) and glutamate (Okumoto et al. 2005). However, no tryptophan-binding PBPs have been described to date, thus an alternative ligand-sensing scaffold was explored for construction of a tryptophan nanosensor.

In γ-proteobacteria like Escherichia coli, transcription of the tryptophan biosynthetic operon is regulated through attenuation (Yanofsky 1981) and inhibitory binding of the tryptophan repressor protein TrpR to the trp operator (Joachimiak et al. 1983), in which binding of L-tryptophan to the repressor results in conformational changes that enhance the repressor's affinity for the operator sequence (Zhang et al. 1987). We have exploited the ligand-induced conformational changes of TrpR for the construction of novel genetically encoded sensors for monitoring of in vivo L-tryptophan levels. It demonstrated the applicability of the metabolite FRET sensor concept to novel ligand-sensing domains and opened up new ways for the construction of nanosensors for metabolites that are only present inside the cell. In addition, a novel strategy was employed for the optimization of the FRET signal, based on the particular topology and conformation of TrpR. The tryptophan nanosensor can be used for the characterization of the dynamics of tryptophan levels in single cells and is compatible with a 96-well screening format. By construction of an additional FRET sensor for hypoxanthine based on the E. coli purine repressor PurR, we further demonstrate the suitability of effector-modulated transcriptional regulators as recognition elements for nanosensors.

In summary, since mammalian cells cannot synthesize tryptophan and depend on its transport across the membrane for the creation of important molecules such as the neurotransmitter serotonin, the hormone melatonin and vitamin B3 niacin, there is a need to develop a method for measuring tryptophan in living cells. The present invention is based in part on the inventors development of a novel genetically-encoded fluorescence resonance energy transfer (FRET) nanosensors for real-time imaging of intracellular tryptophan levels by allosteric coupling of the dimeric E. coli tryptophan repressor (TrpR) to a cyan (eCFP) and yellow (Venus) fluorescent protein FRET pair in various topologies. A twin cassette FRET nanosensor variant consisting of eCFP-TrpR-Venus-TrpR was produced in monkey kidney COS-7 cells for time-resolved monitoring of tryptophan levels in cell cultures in 96-well plates and individual cells. A hypoxanthine FRET sensor based on E. coli PurR demonstrated the generalizability of bacterial repressors as backbones for in vivo sensors. In perfusion experiments with HEK293T cells transfected with genetically-encoded FRET nonosensors, the intracellular tryptophan levels increase during perfusion with tryptophan. The Trp levels decrease during subsequence perfusion with buffer due to efflux and metabolism.

The following examples are provided to describe and illustrate the present invention. As such, they should not be construed to limit the scope of the invention. Those in the art will well appreciate that many other embodiments also fall within the scope of the invention, as it is described hereinabove and in the claims.

### EXAMPLES

Materials and Methods

Chemicals, Strains, Plasmids: All chemicals were of analytical grade and purchased from Sigma-Aldrich. E. coli strains DH5α, TOP10 F′ and BL21(DE3)gold (Stratagene) were used for transformation of Gateway reactions, cloning, and protein production, respectively.

Construction of FLIPW and FLIPpur Sensors: The E. coli trpR gene (Gunsalus et al. 1980) (EcoGene EG11029, TrpR: UniProt P0A881) was amplified from genomic DNA by PCR for cloning in plasmid pGWF1 through pDONR using forward primer (5′-GGGGACAAGTTTGTACAAAAAAGCAGGCTCGGCCCAACAATCACCCTATTCA GC-3′; SEQ ID NO: 11) and reverse primer (5′-GGGGACCACTTTGTACAAGAAAGCTGGGTT ATCGCTTTTCAGCAACACCTCTTC-3′; SEQ ID NO: 12) using the Gateway protocol provided by the manufacturer (Invitrogen). Plasmid pGWF1 is based on the pRSETb expression vector (Novagen) and contains genes for enhanced cyan fluorescent protein (eCFP) and Venus, a yellow fluorescent protein variant, cloned under control of the bacteriophage T7 promoter. Between the gene sequences of eCFP and Venus a chloramphenicol-resistancy gene and lethal ccdB gene are flanked by the necessary attP DNA sequences for insertion of DNA sequences using Gateway cloning technology. The trpR gene was sandwiched between the eCFP and Venus coding sequences resulting in plasmid pTK164. The protein sequence encoded on pTK164 was denoted FLIPW-CTY. By PCR, trpR copies flanked with BamHI or HindIII restriction site sequences were produced. Twin cassette sensor variants were constructed by insertion of trpR copies into pTK164 using unique BamHI and HindIII restriction sites respectively before the ECFP coding sequence (resulting in pTK203) and after the Venus encoding sequence (resulting in pTK204), resulting in sensor variants encoding the repressor dimer in a single gene. A construct in which two trpR copies were connected with a Gly₇ linker was denoted pTK205. The gene products of pTK203, pTK204, and pTK205 were denoted FLIPW-TCTY, FLIPW-CTYT, and FLIPW-CTTY, respectively. The part of the E. coli purR gene (EcoGene EG10800, PurR: Uniprot P0ACP7) encoding amino acid residues 56 to 341 was amplified from genomic DNA by PCR using forward primer (5′-GGTACCGGAGGCGG CGTTAACCACACCAAGTCTATCG-3′; SEQ ID NO: 13) and reverse primer (5′-GGTACCGG CGCCTTTACGACGATAGTCGCGGAACGG-3′; SEQ ID NO: 14) and cloned into pCR4TopoBlunt (Invitrogen). DNA sequencing revealed two T→C mutations at positions 534 and 788, resulting in substitution Leu263Pro (intact PurR numbering). Previously-described affinity mutation Arg190Gln (Lu et al. 1998) was introduced by PCR using primers (5′-GAAATCGGCGTCATCCCCGGCCCGCTGGAACA GAACACCGGCGCAG-3′; SEQ ID NO: 15) and (5′-CTGCGCCGGTGTTCTGTTCCAGCGGGCC GGGGATGACGCCGATTTC-3′; SEQ ID NO: 16). PurR_R190Q was excised from pCR4TopoBluntPurR_R190Q by KpnI and cloned into KpnI-digested pRSET_Flip derived from FLIPrib-250n (Lager et al. 2003), resulting in pFLIPpur encoding a His₆-eCFP-PurR-eYFP fusion protein. FLIPW and FLIPpur constructs were harbored in E. coli BL21(DE3)gold and sensor proteins were produced and purified as described previously (Fehr et al. 2002).

In Vitro Characterization of FLIPW and FLIPpur Sensors: Purified sensor was added to a dilution series of ligand in 20 mM MOPS pH 7.0 (FLIPW) or 20 mM MES pH7.0 (FLIPpur) in the range of 10⁻² to 10⁻⁶ M and analyzed in a monochromator microplate reader (Safire, Tecan, Austria; excitation 433/12 nm, emission 485/12 and 528/12 nm). Protein was diluted to give Venus/eYFP readouts of 20,000 to 30,000 at a manual gain between 70-75. By using the change in FRET ratio upon binding of ligand, binding constants (K_{d}) were determined by fitting the substrate titration curves to a single-site-binding isotherm. Formulas for decrease in ratio upon ligand binding:

R=Rₘₐₓ−(n·[S])/(K_{d}+[S])·(Rₘₐₓ−Rₘᵢₙ)

with [S], substrate concentration; n, number of equal binding sites; R, ratio; Rₘₐₓ, maximum ratio in the absence of ligand; and Rₘᵢₙ, minimum ratio at saturation with ligand. Formula for increase in ratio upon ligand binding:

R=Rₘᵢₙ+(Rₘₐₓ−Rₘᵢₙ)·(n·[S])/(K_{d}+[S])

with [S], substrate concentration; n, number of equal binding sites; R, ratio; Rₘₐₓ, maximum ratio at saturation with ligand; and Rₘᵢₙ, minimum ratio in absence of ligand. Three independent protein preparations were analyzed and each protein preparation was analyzed in triplicate.

Tissue Culture and Transfection: For cytosolic expression in COS-7 cells, the gene encoding CTYT was amplified by PCR with primers encoding unique BamHI and EcoRI restriction sites at the 5′ and 3′ end, respectively, and cloned into BamHI/EcoRI digested pcDNA3.1(+) vector (Invitrogen), resulting in plasmid pTK222. COS-7 cells were grown in Dulbecco's modified Eagle's medium (high glucose; DMEM, Gibco) with 10% fetal calf serum and 50 μg/ml penicillin and streptomycin (Gibco). Cells were cultured at 37° C. and 5% CO2. For imaging, cells were cultured in 8-well LabTekII German tissue culture glass slides (Nalg Nunc International) and transiently transfected at 50-70% confluence using Lipofectamine 2000 Reagent (Invitrogen) in Opti-MEM I reduced serum medium (Gibco). After transfection, cells were cultured for 16 hours in Opti-MEM followed by 3 hours in DMEM prior to imaging. Transfection efficiency as determined by counting fluorescing cells was at least 30%.

Microplate Assays: Adherent cells in 96-well microplates were washed once with 100 μl Tyrode's buffer (119 mM NaCl, 2.5 mM KCl, 2 mM CaCl₂, 2 mM MgCl₂, 25 mM HEPES, 30 mM glucose, pH 7.3-7.4). The initial FRET ratio was measured by recording the eCFP and Venus emissions at 485 nm and 528 nm, respectively, after excitation of eCFP at 433 nm in a Safire monochromator microplate reader (Tecan, Grodig, Austria). Standard deviation of the initial ratios was less than 10%. After addition of 100 μl tryptophan in Tyrode's buffer the FRET ratio was recorded with 2 min intervals for up to two hours. Uptake rates were determined from linear parts in the initial FRET change and fitted with the non-linear regression program Origin 6.1 (OriginLab, Northhampton, Me., USA).

Imaging: Ratio imaging was performed on an inverted fluorescence microscope (DM IRE2, Leica) with a CoolSnap HQ digital camera (Roper) and 20× immersion Corr, 40× oil, or 63× water immersion lenses (HC PL APO 20×/0.7 or HCX PL APO, Leica, Germany). Dual emission intensity ratios were simultaneously recorded using a DualView with an OI-5-EM filter set (eCFP 480/30; eYFP 535/40; Optical 17 Insights, USA) and Metafluor 6.3r7 software (Molecular Devices, USA). Excitation was provided by a Sutter Instruments Lambda DG4. Images were acquired within the linear detection range of the camera and depending on the expression level, exposure times varied between 50 to 200 ms, with software binning between 2 and 3. Fluorescence intensities for eCFP and Venus were typically in the range of 1500-2000 and 2500-3000, respectively. Typical background values were around 100. Cells were perfused with Tyrode's buffer at flow rates of 1.0 ml/min in a chamber with a total volume of 0.5 ml. Analyses were repeated at least three times with similar results.

3D Modeling of FLIPTrpR Variants: Structural models of FLIPW sensors were constructed using the crystal structures of Trp repressor in complex with L-Trp (PDB identifier 1WRP; PDB: Protein data bank at http://www.rcsb.org/pdb/home/home.do) and Venus (PDB identifier 1 MYW). Proteins were manually docked in the various topologies using MAGE (kinemage.biochem.duke.edu).

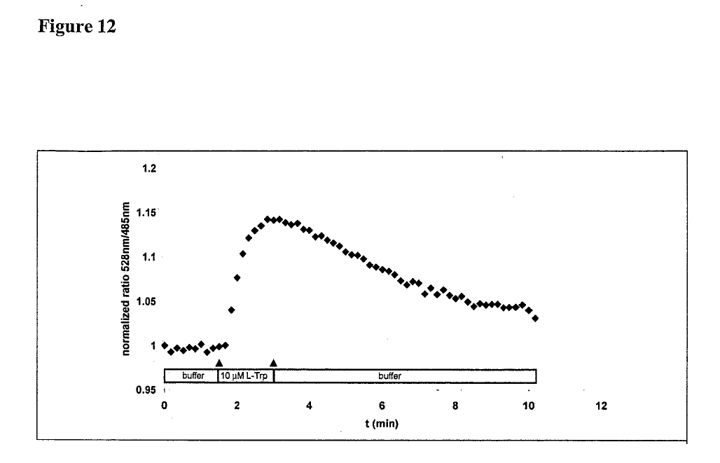

Perfusion of HEK293T cells transfected with pTK222: Cells were perfused with Tyrode's buffer. Between 1′30″ and 3′ (indicated by triangles in FIG. 12) buffer was supplemented with 10 μM L-tryptophan. Response of the sensor is determined from the ratio of fluorescence output at 528 nm and 485 nm. During perfusion with tryptophan, the intracellular tryptophan levels increase. The Trp levels decrease during subsequent perfusion with buffer due to efflux and metabolism.

### Example 1

### A Ligand-Binding Scaffold for L-Tryptophan

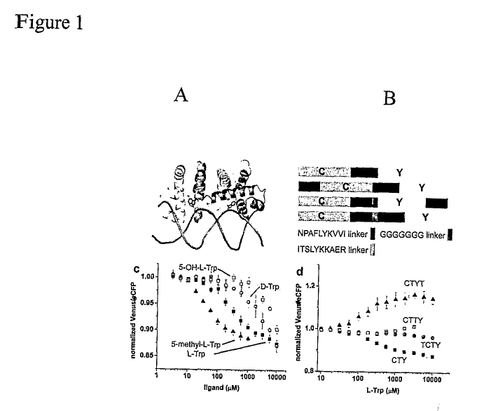

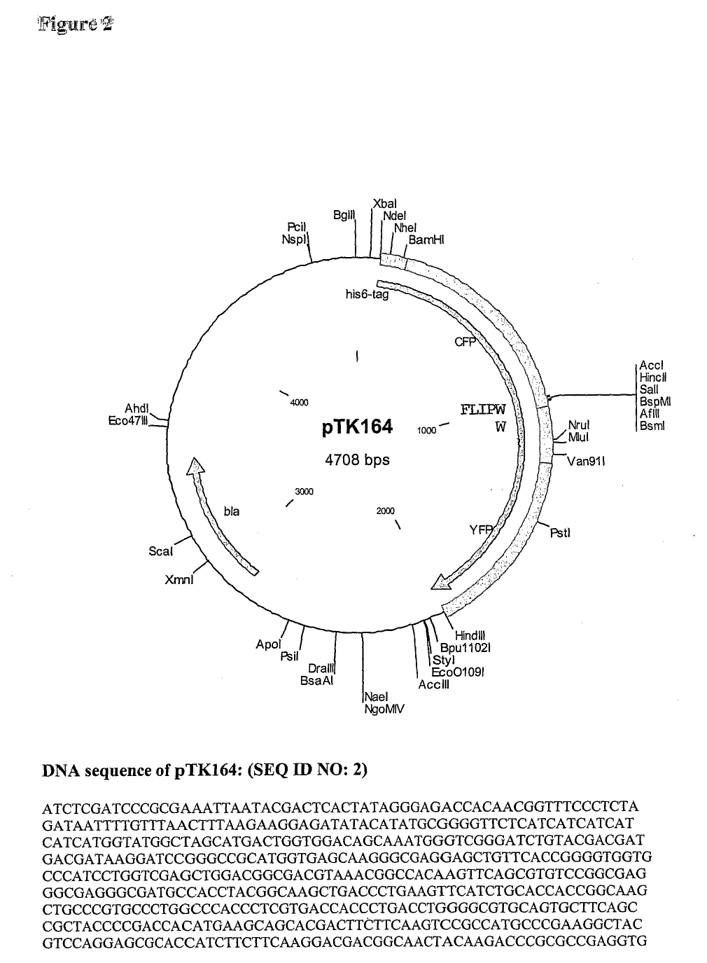

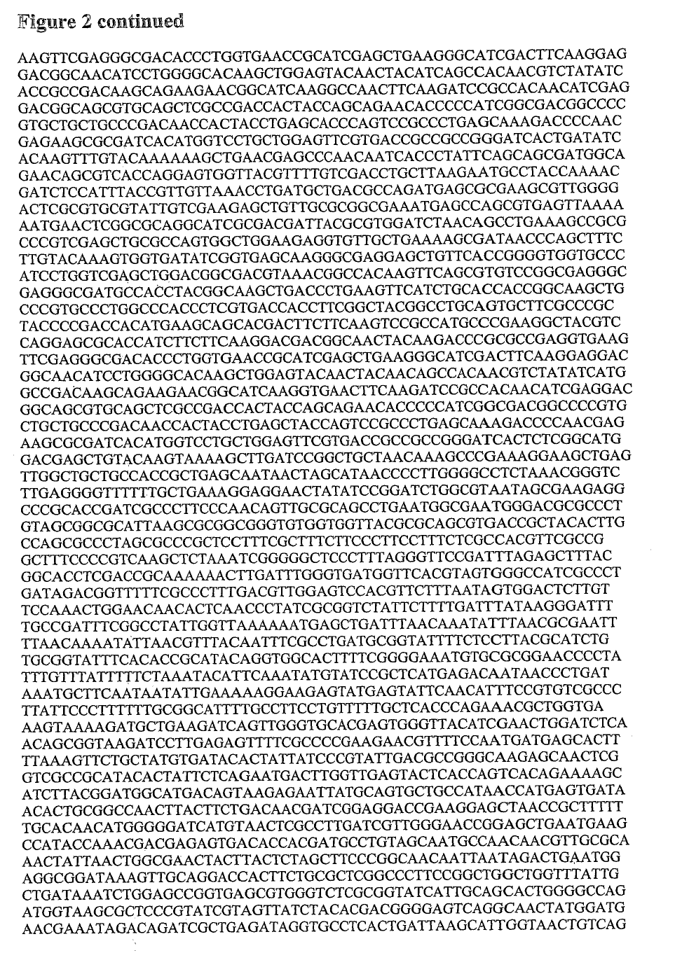

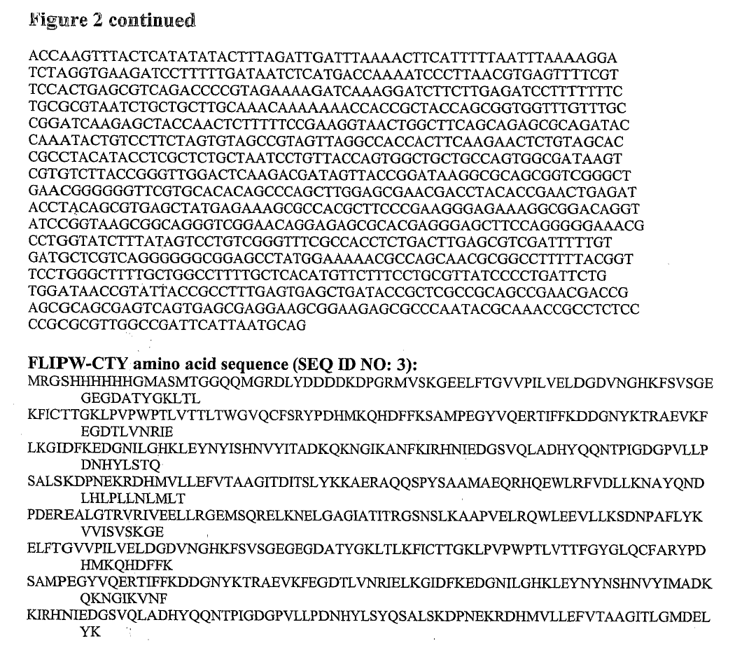

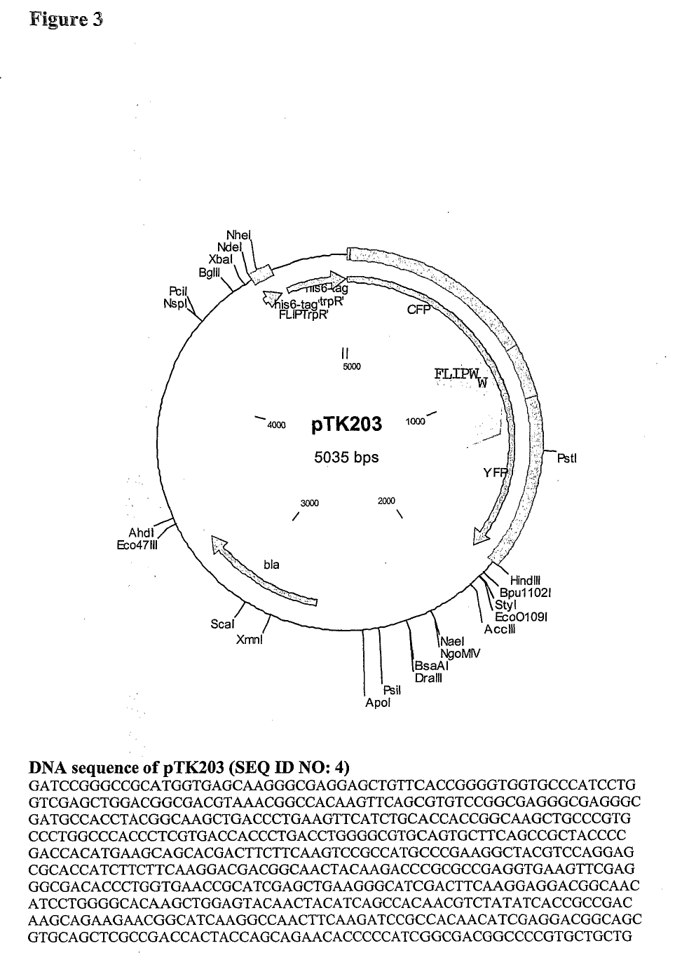

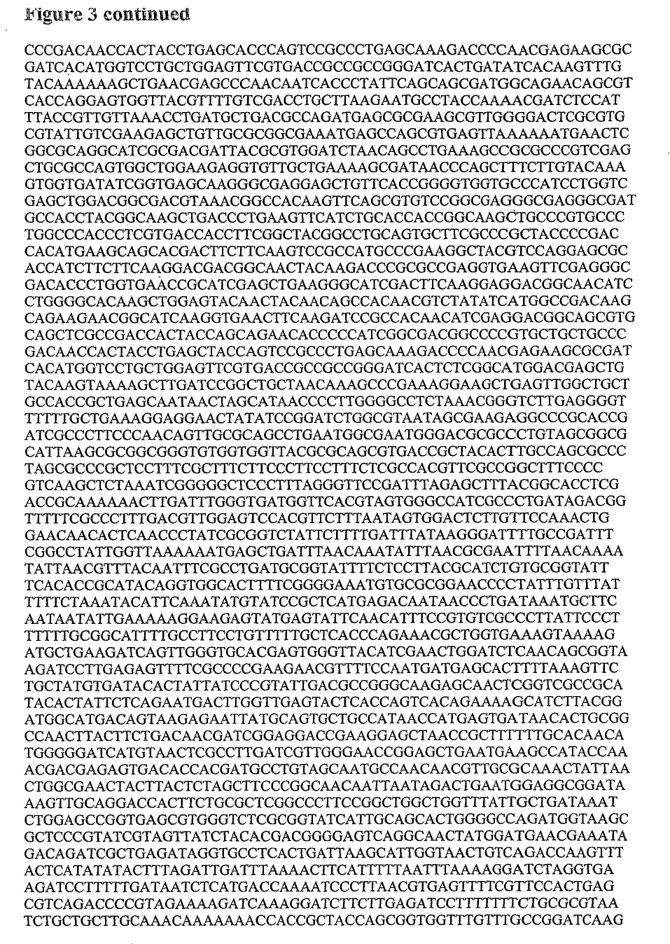

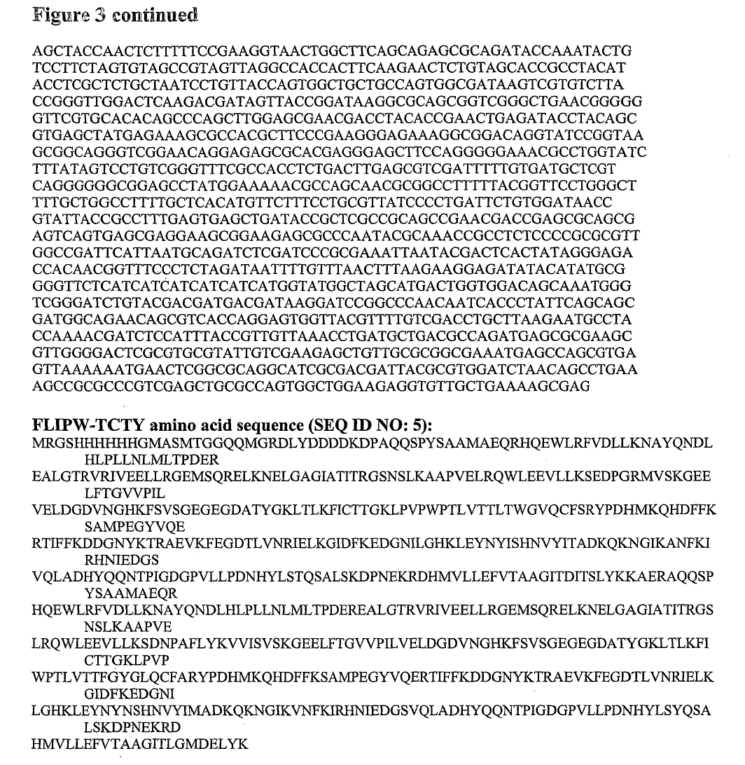

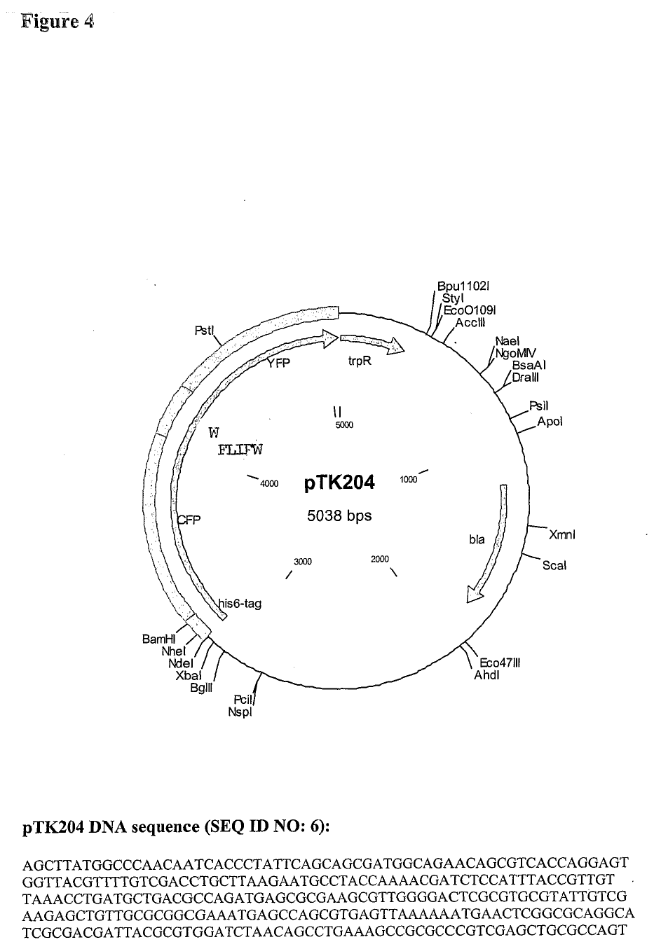

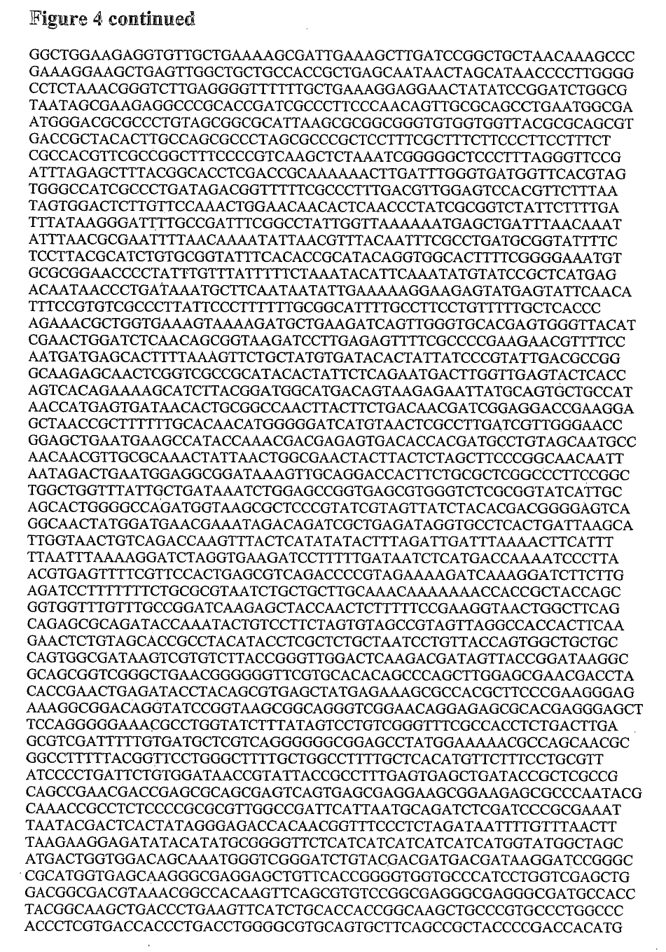

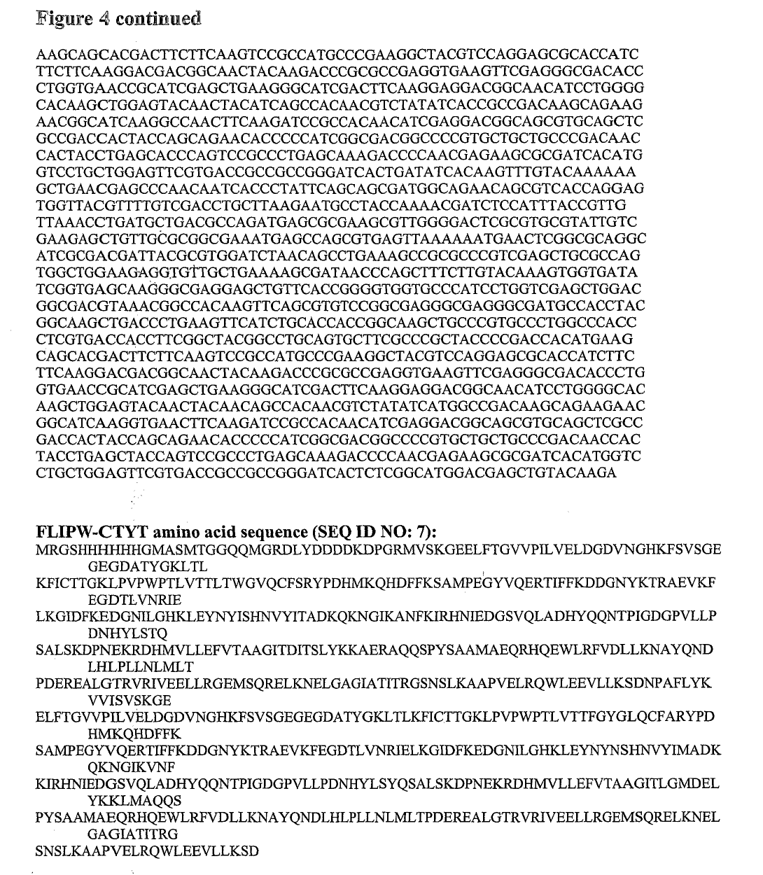

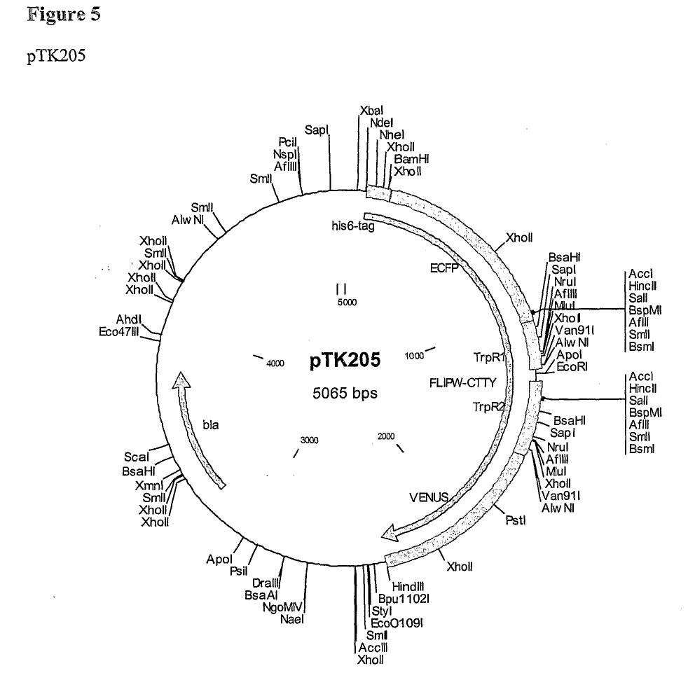

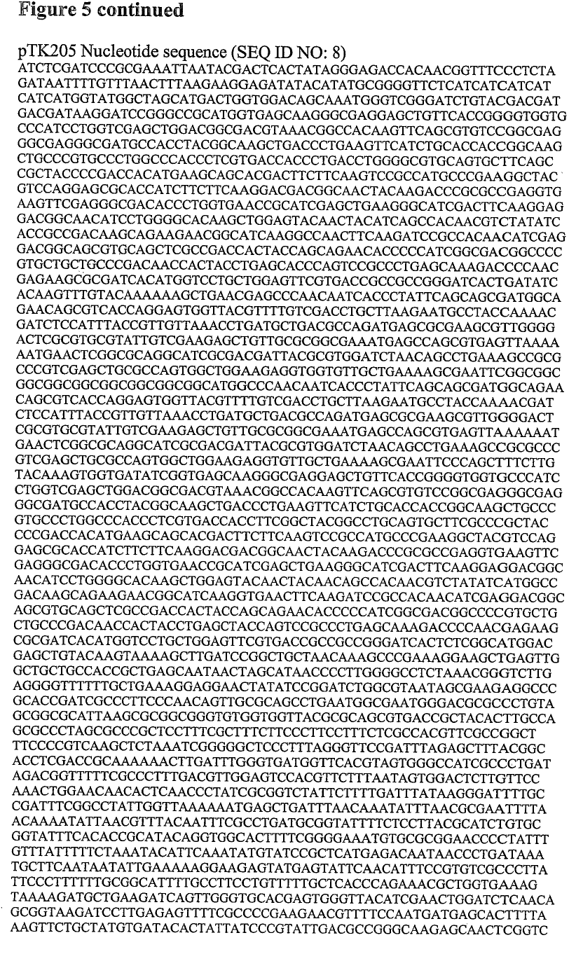

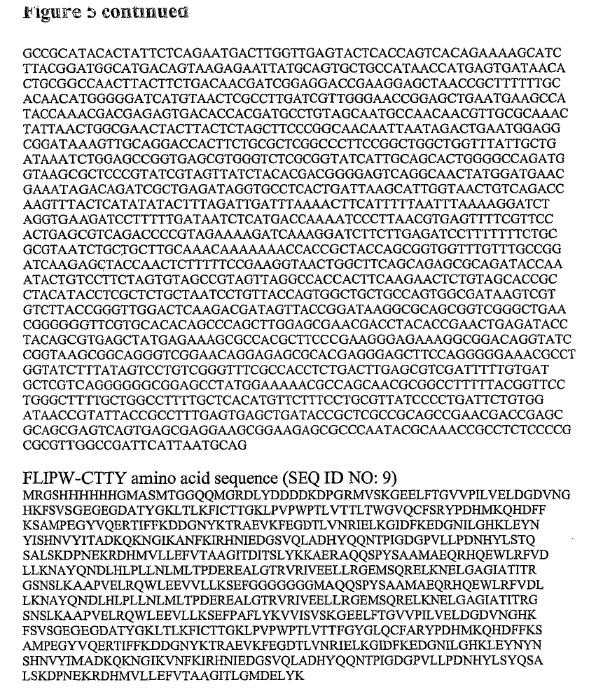

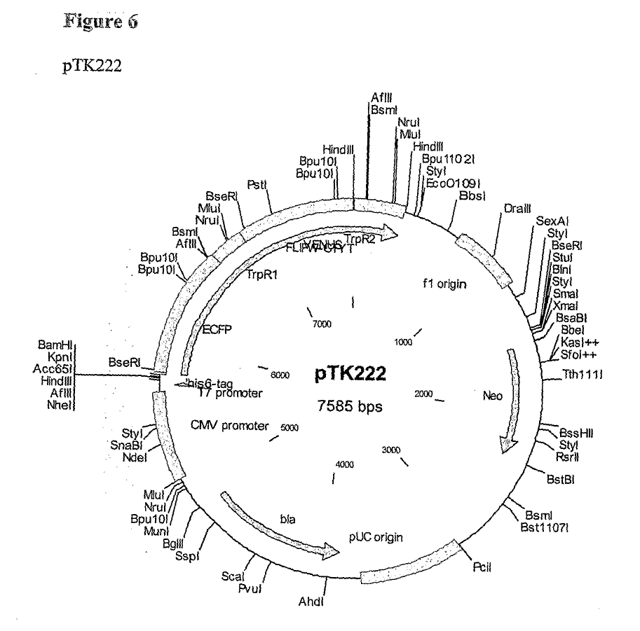

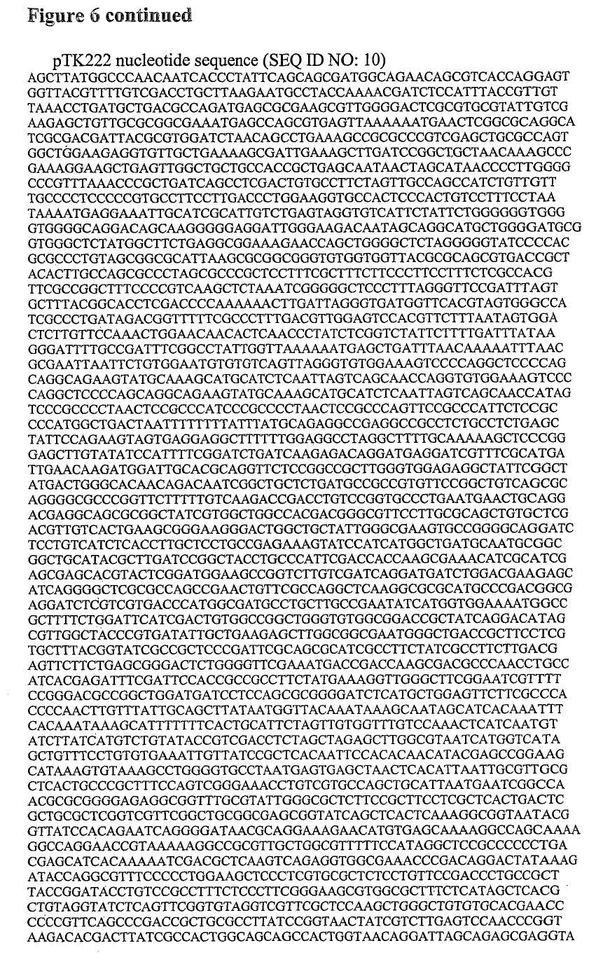

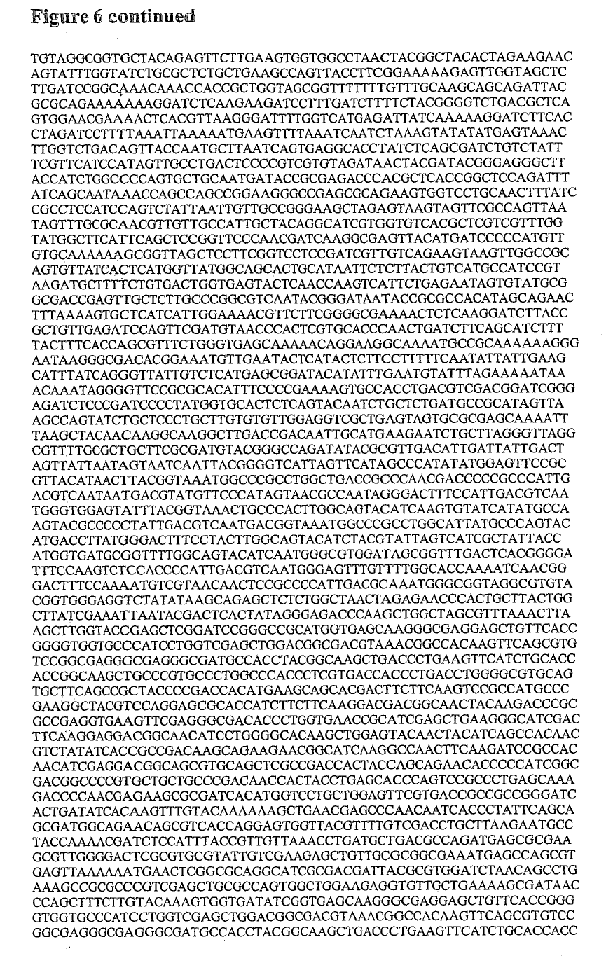

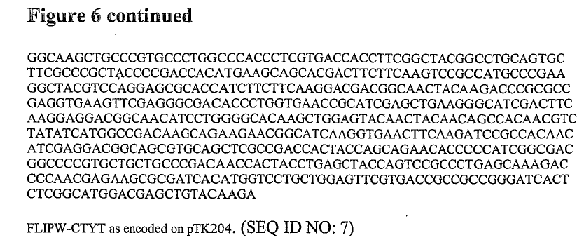

In this Example, the tryptophan operon repressor TrpR is the ligand binding protein moiety used to generate biosensors. The E. coli tryptophan operon repressor TrpR is an all-helical protein of 108 amino acids organized into 6 α-helices that selectively binds L-tryptophan with micromolar affinity (FIG. 1A) (Marmorstein et al. 1987). The active conformation of TrpR is a dimer in which 5 of the 6 helices are involved in intermolecular contacts (Schevitz et al. 1985). With both chains contributing to the tryptophan-binding sites, two TrpR molecules are necessary to form two functional sites (FIG. 1A). In the absence of tryptophan, a part of TrpR is unfolded (Reedstrom et al. 1995), which likely corresponds to the helix-turn-helix motifs that form the ‘DNA-reading heads’, since they undergo structural rearrangements upon binding of tryptophan in TrpR crystal structures (Zhang et al. 1987) and their flexibility is essential for the recognition of operator sequences (Gryk et al. 1996). In addition, tryptophan binding results in a shift of the relative distance and orientation of the N- and C-termini of the repressor with respect to one another (Zhang et al. 1987), which was detected as a change in fluorescence resonance energy transfer (FRET) between fused fluorophores. The E. coli tryptophan repressor gene was sandwiched between eCFP and Venus coding sequences (FIG. 1B). Production of the translated fusion product FLIPW-CTY (CTY=eCFP-TrpR-VenusYFP) could be readily detected by recording the emission spectrum of the eCFP-Venus FRET signal in whole cell cultures. Indeed, addition of tryptophan decreased the FRET efficiency of the purified protein in vitro, which was visible as a decrease in Venus fluorescence intensity (FIG. 1C) (see Table 1 for details). FLIPW-CTY bound L-tryptophan with an apparent K_{d} of 220±20 μM, which is about an order of magnitude larger than free TrpR (Marmostein et al. 1987). Titration of FLIPW-CTY with D-tryptophan resulted in a decrease of FRET ratio at about 5-fold higher concentrations than L-tryptophan, thus controlling for the possibility that the decrease in FRET ratio of FLIPW-CTY resulted from quenching of the chromophores (FIG. 1C). Analogous to wild-type TrpR, FLIPW-CTY binds ligands in order of decreasing affinity: L-5-methyl-tryptophan>L-tryptophan>D-tryptophan>L-5-hydroxy-tryptophan (see Supporting Table 2 for details) (Marmostein et al. 1987).

TABLE 1
Signal Change and L-Tryptophan Affinities of FLIPW Sensors

| Sensor | Apo ratio* | Δratio | Δratio (%) | K_{d} (μM) |
|---|---|---|---|---|
| FLIPW-CTY | 4.10 | −0.41 | −10 | 220 ± 20 |
| FLIPW-TCTY | 1.57 | −0.03 | −2 | 20 |
| FLIPW-CTYT | 2.08 | 0.35 | 17 | 210 ± 20 |
| FLIPW-CTTY | 2.85 | n.d.^{†} | n.d.^{†} | n.d.^{†} |

*ratio defined as fluorescence intensity quotient of emission at 528 nm/485 nm
^{†}n.d. not determined

TABLE 2
Affinities of FLIPW-CTY and FLIPW-CTYT for Tryptophan
Substrates (mM)

| Substrate | FLIPW-CTY | FLIPW-CTYT |
|---|---|---|
| L-tryptophan | 0.22 ± 0.02 | 0.21 ± 0.02 |
| D-tryptophan | 3.1 ± 0.3 | n.d.* |
| L-5-methyl-tryptophan | 0.06 ± 0.01 | 0.06 ± 0.02 |
| L-5-hydroxy-tryptophan | 6.0 ± 0.8 | n.d.* |

*n.d. not determined

### Example 2

### Twin-Cassette FLIPW Nanosensor Variants

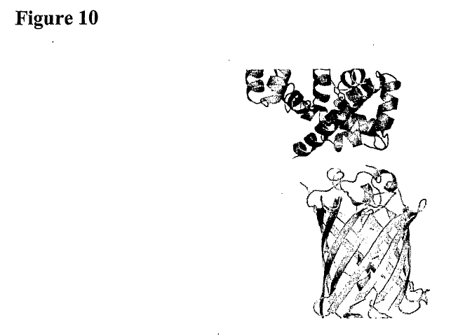

As a result of the unique conformational properties of TrpR, the functional FLIPW-CTY sensor consisted of two TrpR molecules and four fluorophores tightly packed together, which potentially could affect the binding affinity due to steric hindrance or result in signal loss due to averaging of the fluorophore signals. It was rationalized that sensors with a single fluorophore pair would have improved sensing characteristics, and therefore the three possible sensors containing two TrpR copies in a single gene product (treating the two fluorophores as equivalent) were constructed (FIG. 1B). In variants FLIPW-TCTY and FLIPW-CTYT the termini of the green fluorescent protein variants span the distance between the N- and C-termini of the respective TrpR molecules in the dimer (˜22 Å, see FIG. 10). One of the fluorophores in these variants should therefore be rotationally restrained by these attachment points, which could improve signal change due to decreased conformational averaging (Deuschle et al. 2005; Van der Meer et al. 1994). For the construction of FLIPW-CTTY, two copies of the repressor gene were connected by a flexible linker consisting of 7 glycine residues and inserted between the fluorophores. This linker was designed to loosely connect the two TrpR proteins without changing the dimer conformation and was based on a model constructed in Modeller8v1 (Marti-Renom et al. 2000). The FRET ratio of FLIPW-CTTY and FLIPW-TCTY changed only slightly when titrated with L-tryptophan (FIG. 1D). The apparent binding constant of FLIPW-TCTY for L-tryptophan was around 20 μM, which is comparable to wild-type TrpR (Marmorstein et al. 1987). The ratio change of FLIPW-CTTY was not uniform, slightly decreasing around 20 μM L-Trp similar to FLIPW-TCTY and increasing above 1 mM. Interestingly, when FLIPW-CTYT was titrated with L-tryptophan an increase in FRET ratio was observed, indicating a significant change in chromophore orientation with respect to FLIPW-CTY. The absolute FRET ratio of FLIPW-CTYT was half that of FLIPW-CTY (see Table 1). FLIPW-CTYT bound L-tryptophan with an apparent affinity of 210±20 μM. This made the sensor suitable for monitoring physiological tryptophan levels in mammalian cells (˜25 μM-2 mM).

### Example 3

### Molecular Modeling of FLIPW Sensors

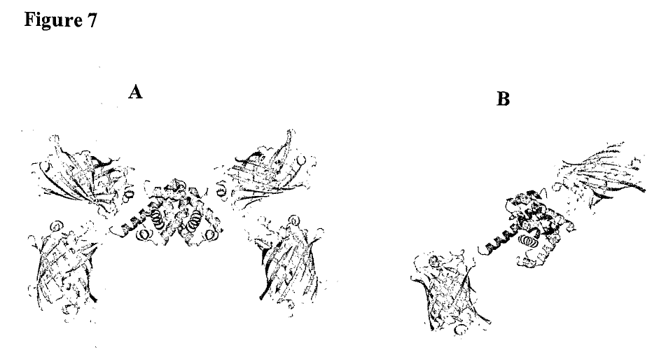

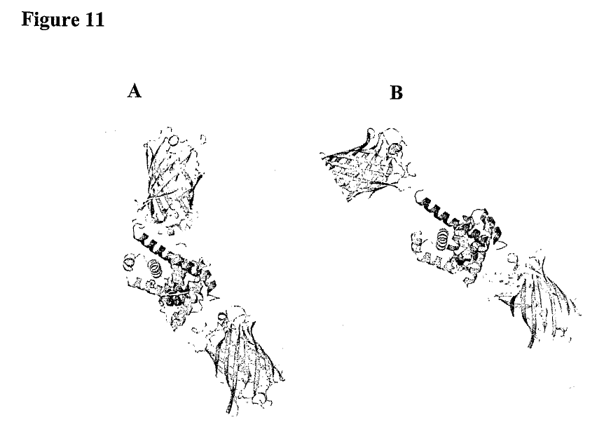

In this Example, molecular modeling was performed to explain the observed FRET signal changes. The original sensor, FLIPW-CTY, is predicted to dimerize, resulting in two pairs of eCFP-Venus fluorophores close to one another on either side of the TrpR dimer (FIG. 7A). This sensor showed the highest FRET efficiency and a significant FRET decrease upon ligand binding. The FLIPW-CTYT sensor is modeled to form the functional TrpR dimer intra-molecularly, resulting in a single eCFP and a single Venus molecule per sensor (FIG. 7B). This sensor has a lower initial FRET ratio, consistent with the greater distance between the fluorophore dipoles, but the relative FRET change is higher, perhaps due to the rigidification of the Venus molecule by its fusion to both TrpR monomers. The FLIPW-CTTY and FLIPW-TCTY sensors do not show sufficient ligand-dependent ratio changes to be useful as sensors. Molecular modeling explains the surprising disparity between the FLIPW-CTYT and FLIPW-TCTY sensors, due to the inequivalence of the TrpR termini (FIG. 11).

### Example 4

### Tryptophan Uptake in COS-7 Cell Cultures in 96-Well Microplates

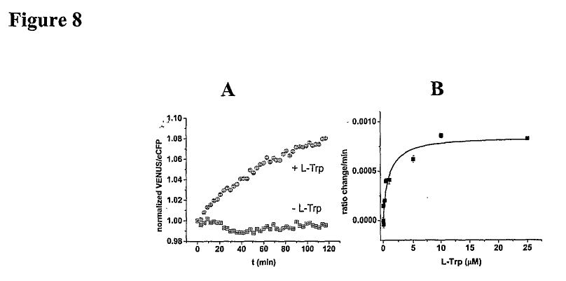

In this Example, tryptophan uptake in COS-7 cell cultures in 96-well microplates were investigated. COS-7 cell cultures seeded in a 96-well microplate were transiently transfected with pTK222 for cytosolic production of FLIPW-CTYT sensor. Microscopic analysis of transfected cells showed that FLIPW-CTYT was produced exclusively in the cytosol and did not enter the nucleus similar to results obtained with the glucose FRET sensor in COS-7 cells (Fehr et al. 2003) (data not shown). When microwell-grown cells expressing FLIPW-CTYT were incubated in Tyrode's buffer containing tryptophan and analyzed in a microplate reader, an increase in FRET ratio was observed indicating an increase in cytosolic tryptophan levels as a result of uptake (FIG. 8A). The rate in FRET increase depended on the external tryptophan concentration and showed Michaelis-Menten type kinetics with an apparent enzymatic specificity constant K_{M} of 0.88±0.27 μM for combined transport and metabolism (FIG. 8B). The FLIPW-CTYT sensor is thus suitable to study factors that influence tryptophan transport and metabolism and can be used in high-throughput fluorescence-based assay systems.

### Example 5

### A Hypoxanthine Sensor Based on E. coli PurR

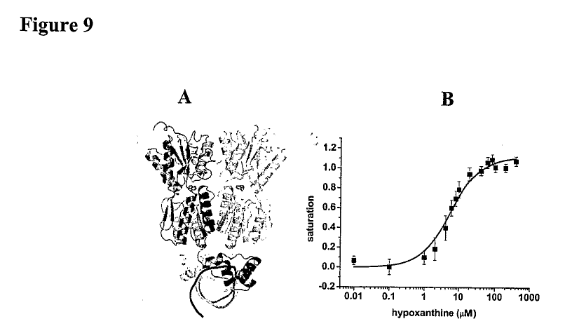

The successful use of the E. coli tryptophan repressor as a recognition element for FRET nanosensors encouraged further exploration of transcriptional regulators as ligand-sensing domains. The LacI/GalR family of repressors comprises PBP-related effector-binding domains that bind a wide variety of corepressors i.e. lactose (LacI), fructose (Vibrio cholera VCA0519), trehalose-6-phosphate (TreR), and purines (PurR) (Fukami-Kobayashi et al. 2003). The latter E. coli purine repressor PurR controls expression of genes encoding enzymes for de novo synthesis of purines (Meng et al. 1990). PurR has a bipartite structure with a DNA-binding N-terminal domain and a larger corepressor-binding C-terminal domain (FIG. 9A) (Schumacher et al. 1994). Binding of the end products hypoxanthine or guanine to PurR introduces conformational changes (Choi et al. 1992). The C-terminal part of PurR Arg190Gln (Lu et al. 1998) was sandwiched between eCFP and eYFP and the resulting FLIPpur sensor showed a 3% decrease in FRET ratio in the presence of hypoxanthine with an apparent K_{d} of 5.6±0.7 μM (FIG. 9B), which is identical to that of free PurR Arg190Gln (Lu et al. 1998). For in vivo use of FLIPpur, the current signal-to-noise ratio will need to be improved by employing previously reported strategies (Deuschle et al. 2005). This result shows that the ligand-binding domains of the LacI/GalR repressor family provide additional scaffolds for the construction of FRET nanosensors with novel specificities.

### Example 6

### Perfusion of HEK293T Cells Transfected with pTK222

Cells were perfused with Tyrode's buffer. Between 1′30″ and 3′ (indicated by triangles in FIG. 12) buffer was supplemented with 10 μM L-tryptophan. Response of the sensor is determined from the ratio of fluorescence output at 528 nm and 485 nm. During perfusion with tryptophan, the intracellular tryptophan levels increase. The Trp levels decrease during subsequent perfusion with buffer due to efflux and metabolism.

### DISCUSSION

Mammalian cells cannot synthesize the amino acid tryptophan and rely on its transport across the plasma membrane for basic cell functioning. Tryptophan is necessary for protein synthesis, as it accounts for 1.3% of the amino acids in human proteins. Furthermore, tryptophan is the precursor of other vital molecules like serotonin, melatonin and niacin.

The FLIPW nanosensors described in this study allow for non-invasive real-time spatio-temporal imaging of intracellular tryptophan levels and flux, offering advantages over conventional analysis methods. The properties of the E. coli transcriptional regulator TrpR have been employed as the recognition element for the construction of FRET sensors for tryptophan. As noted previously, the use of bacterial proteins for the construction of intracellular sensors reduces the problem of cross-interference with endogenous metabolic and signal transduction pathways in eukaryotic cells (Belousov et al. 2006). Genetically-encoded nanosensors further offer the advantage of subcellular sensor targeting through judicious choice of leader sequences i.e, nuclear- and ER-targeted glucose nanosensors (Fehr et al. 2004; Fehr et al. 2005) and cell-surface display of a glutamate nanosenor (Okumoto et al. 2005).

Most FRET nanosensors have been based on the ligand-binding-induced Venus-fly-trap-like conformational changes of bacterial periplasmatic binding proteins (PBPs) (Fehr et al. 2003; Fehr et al., 2002; Lager et al. 2003; Okumoto et al. 2005), which consist of two well-structured lobes with the ligand-binding site located at the interface. TrpR is about three times smaller than the average PBP and partially unfolded in the absence of tryptophan (Reedstrom et al. 1995). In the presence of tryptophan the protein adopts the conformation observed in crystal structures (Zhang et al., 1987) and the concomitant conformational changes allow for the detection of tryptophan binding by FRET. The FLIPW sensors, therefore, represent a novel class of nanosensors.

The ligand-binding affinities of the PBP-based FRET nanosensors are typically similar to those of the free PBPs (Fehr et al. 2003; Fehr et al., 2002; Lager et al. 2003; Okumoto et al. 2005), at least for linearly-fused fluorophores. In the case of FLIPW sensors, the situation is more complex, since the two binding sites are formed at the interface of the dimer. It is thus possible that the addition of fluorophores may affect ligand-induced folding and, as such, formation of the tryptophan-binding sites. Indeed, fusion of the tryptophan repressor to eCFP and Venus resulted in 10-fold reduced affinities of the CTY nanosensor for all tested tryptophan substrates compared to reported dissociation constants for TrpR (Marmorstein et al. 1987). Fortuitously, the addition of the fluorophores resulted in a sensor dynamic range suitable for detection of cytosolic tryptophan levels.

FRET has been a successful reporter signal for small molecule sensors (Lalonde et al. 2005; De et al. 2005). According to the Förster theory, the efficiency of the energy transfer depends on the distance between the fluorophores and their dipole orientation (Jares-Erijman et al 2003). These small molecule sensors can be engineered by modification of linker sequences between reporter and sensing domains and/or insertion of fluorophores in surface loops of the sensing domain, resulting in increased and/or reversed signal outputs of FRET nanosensors (Deuschle et al. 2005). Since TrpR dimerizes to form its ligand-binding sites, we applied a novel approach for engineering of the FRET signal. Insertion of a second TrpR coding sequence in the principal FLIPW-CTY changed the FRET output depending on the position of the insertion site. While insertion before eCFP and between eCFP and Venus largely killed the FRET response, a TrpR copy after eCFP reversed the FRET response and increased the ratio change. Comparison of structural models of the FLIPW-CTY and FLIPW-CTYT sensors predicted that the fluorophores would be closer together in the former. Since FRET efficiency is inversely correlated with the distance between the fluorophores as described in the Förster equation (Jares-Erijman et al. 2003), the experimentally determined FRET ratio and the models are consistent with each other.

FLIPW-CTYT was used for monitoring tryptophan uptake in cell cultures grown in 96-well microtiter plates. Based on the measurements, the effective K_{M} for combined tryptophan uptake and metabolism in COS-7 cells was in the micromolar range. Previously, transport systems for tryptophan, B⁰ (Broer et al. 2004), TAT1 (Kim et al. 2002), LAT1 (Kanai et al. 1998), and LAT2 (Rossier et al. 1999; Segawa et al. 1998; Pineda et al. 1999), had been characterized by uptake of radiolabeled amino acids in Xenopus oocytes. Tryptophan uptake affinities have only been determined for human TAT1 (450 μM) (Kim et al. 2002) and LAT2 (58 μM) (Pineda et al. 1999) and relate to the sum of intracellular pools of free, incorporated, and degraded tryptophan in oocytes. Affinities obtained using FLIPW-CTYT, on the other hand, are more specific as they have been determined for the pool of free tryptophan in the targeted subcellular compartment. The new sensor thus provides a complementary tool for monitoring steady state levels, uptake, counterexchange and will be an important tool for analyzing the factors that control tryptophan flux in living cells. High-throughput assays can be devised in which the effect of drugs or siRNAs is tested systematically (Myers et al. 2003).

In perfusion experiment with HEK293T cells transfected with FLIPW-CTYT, the intracellular tryptophan levels increase during perfusion with tryptophan. The Trp levels decrease during subsequent perfusion with buffer due to efflux and metabolism.

The PBP family encompasses a wide variety of ligand specificities including carbohydrates, amino acids, anions, metal ions, dipeptides and oligopeptides, and has up to now yielded sensing domains of FRET nanosensors for maltose (Fehr et al. 2002), glucose (Fehr et al. 2003), ribose (Lager et al. 2003) and glutamate (Okumoto et al. 2005). As the FLIPW sensors demonstrate, other protein scaffolds that undergo conformational changes upon ligand binding can provide sensing domains for nanosensors with specificities that are not represented in the PBP family, such as tryptophan. E. coli tryptophan repressor TrpR is not part of a protein family with different substrate specificities, which could be used for the expansion of the current set of nanosensors. However, the wealth of bacterial transcriptional regulators, which change affinity for operator sequences upon binding of effectors, may provide potential sensing domains for novel FRET metabolite nanosensors. As an example, we constructed FLIPpur, a hypoxanthine FRET sensor, based on the effector-binding domain of E. coli PurR of the LacI/GalR family of repressors. In addition, a fluorescent sensor for hydrogen peroxide, HyPer, was recently constructed by insertion of circularly permuted yellow fluorescent protein in the H₂O₂-sensing part of bacterial transcriptional regulator OxyR (Belousov et at. 2006). Among the effectors to which transcriptional regulators have been evolved to respond to are many molecules that are desirable to monitor in light of metabolic imaging or their medical relevance e.g. salicylate (Pseudomonas sp. NahR) (Park et al. 2005), nucleosides (E. coli XapR) (Jorgensen et al. 1999), and tetracycline (TetR) (Orth et al. 2000). Alternatively, novel ligand specificities may be engineered (Galvao et al. 2006).

All publications, patents and patent applications discussed herein are incorporated herein by reference. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### REFERENCES

Belousov, V. V., Fradkov, A. F., Lukyanov, K. A., Staroverov, D. B., Shakhbazov, K. S., Terskikh, A. V. & Lukyanov, S. (2006) Nat Methods 3:281-286.
Broer, A., Klingel, K., Kowalczuk, S., Rasko, J. E., Cavanaugh, J., Broer, S. & School of, B. (2004) J Biol Chem 279:24467-24476.
Broer, S., Cavanaugh, J. A. & Rasko, J. E. J. (2005) Biochem Soc Trans 33, 233-236.
Choi, K. Y. & Zalkin, H. (1992) J Bacteriol 174, 6207-6214.
De, S., Macara, I. G. & Lannigan, D. A. (2005) J Steroid Biochem Mol Biol 96:235-244.
Deuschle, K., Okumoto, S., Fehr, M., Looger, L. L., Kozhukh, L. & Frommer, W. B. (2005) Protein Sci 14:2304-2314.
Fehr, M., Lalonde, S., Ehrhardt, D. W. & Frommer, W. B. (2004). “Live imaging of glucose homeostasis in nuclei of COS-7 cells.” Journal of fluorescence. 14(5):603-609.
Fehr, M., Lalonde, S., Lager, I., Wolff, M. W., and Frommer, W. B. (2003) “In vivo imaging of the dynamics of glucose uptake in the cytosol of COS-7 cells by fluorescent nanosensors.” J. Biol. Chem. 278:19127-19133.
Fehr, M., Takanaga, H., Ehrhardt, D. W. & Frommer, W. B. (2005) Mol Cell Biol 25:11102-11112.
Fehr, M., W. B. Frommer & Lalonde, S. (2002). “Visualization of maltose uptake in living yeast cells by fluorescent nanosensors.” Proc Natl Acad Sci USA 99(15):9846-9851.
Fukami-Kobayashi, K., Tateno, Y. & Nishikawa, K. (2003) Mol Biol Evol 20:267-277.
Galvao, T. C. & de Lorenzo, V. (2006) Curr Opin Biotechnol 17:34-42.
Gryk, M. R., Jardetzky, O., Klig, L. S. & Yanofsky, C. (1996) Protein Sci 5:1195-1197.
Gu, C., J. J. Cali, D. M. F. Cooper, (2002). “Dimerization of mammalian adenylate cyclases” European Journal of Biochemistry 269: 413-421.
Gunsalus, R. P. and Yanofsky, C. (1980). “Nucleotide sequence and expression of Escherichia coli trpR, the structural gene for the trp aporepressor.” Proc Natl Acad Sci USA. 77(12):7117-7121.
Jares-Erijman, E. A. & Jovin, T. M. (2003) Nat Biotechnol 21:1387-1395.
Joachimiak, A., Kelley, R. L., Gunsalus, R. P., Yanofsky, C. & Sigler, P. B. (1983) Proc Natl Acad Sci USA 80(3), 668-672.
Jorgensen, C. & Dandanell, G. (1999) J Bacteriol 181, 4397-4403.
Kanai, Y., Segawa, H., Miyamoto, K., Uchino, H., Takeda, E. & Endou, H. (1998) J Biol Chem 273:23629-23632.
Karasawa S, Araki T, Nagai T, Mizuno H, Miyawaki A. Cyan-emitting and orange-emitting fluorescent proteins as a donor/acceptor pair for fluorescence resonance energy transfer. Biochem J. 2004 381:307-312.
Kim, D. K., Kanai, Y., Matsuo, H., Kim, J. Y., Chairoungdua, A., Kobayashi, Y., Enomoto, A., Cha, S. H., Goya, T. & Endou, H. (2002) Genomics. 79:95-103.
Lager, I., Fehr, M., Frommer, W. B. & Lalonde, S. (2003). “Development of a fluorescent nanosensor for ribose.” FEBS Lett 553(1-2):85-89.
Lalonde, S., Ehrhardt, D. W. & Frommer, W. B. (2005) Curr Opin Plant Biol 8:574-581.
Liu, J., K. S. Knappenberger,. K. Helena, A. Gunilla, E. Nilsson, C. Dartsch, C. W. Scott, et al. (2003). “A homogeneous in vitro functional assay for estragon receptors: coactivator recruitment” Molicular Endocrinology 17(3): 346-355.
Lu, F., Schumacher, M. A., Arvidson, D. N., Haldimann, A., Wanner, B. L., Zalkin, H. & Brennan, R. G. (1998) Biochemistry 37, 971-982.
Marmorstein, R. Q., Joachimiak, A., Sprinzl, M. & Sigler, P. B. (1987). “The structural basis for the interaction between L-tryptophan and the Escherichia coli trp aporepressor.” The Journal of biological chemistry. 262(10): 4922-4927.
Marmorstein, R. Q., P. B. Sigler, et al. (1989). “Stereochemical effects of L-tryptophan and its analogues on trp repressor's affinity for operator-DNA.” The Journal of biological chemistry. 264(16): 9149-9154.
Marti-Renom, M. A., Stuart, A. C., Fiser, A., Sâanchez, R., Melo, F. & Sali, A. (2000) Ann Rev Bioph Biomol Struct. 29, 291-325.
Meng, L. M., Kilstrup, M. & Nygaard, P. (1990) Eur J Biochem 187, 373-379.
Myers, J. W., Jones, J. T., Meyer, T. & Ferrell, J. E., Jr. (2003) Nat Biotechnol 21, 324-328.
Okumoto, S., Looger, L. L., Micheva, K. D., Reimer, R. J., Smith, S. J. & Frommer, W. B. (2005). “Detection of glutamate release from neurons by genetically encoded surface-displayed FRET nanosensors.” Proceedings of the National Academy of Sciences of the United States of America. 102(24): 8740-8745.
Orth, P., Schnappinger, D., Hillen, W., Saenger, W. & Hinrichs, W. (2000) Nat Struct Biol 7, 215-219.
Otwinowski, Z., Schevitz, R. W., Zhang, R. G., Lawson, C. L., Joachimiak, A., Marmorstein, R. Q., Luisi, B. F. & Sigler, P. B. (1988). “Crystal structure of trp repressor/operator complex at atomic resolution.” Nature. 335(6188): 321-329.
Park, H. H., Lee, H. Y., Lim, W. K. & Shin, H. J. (2005) Arch Biochem Biophys 434, 67-74.
Pineda, M., Fernandez, E., Torrents, D., Estevez, R., Lopez, C., Camps, M., Lloberas, J., Zorzano, A. & Palacin, M. (1999) J Biol Chem 274, 19738-19744.
Reedstrom, R. J. & Royer, C. A. (1995) J Mol. Biol. 253, 266-276.
Rossier, G., Meier, C., Bauch, C., Summa, V., Sordat, B., Verrey, F. & Kuhn, L. C. (1999) J Biol Chem 274, 34948-34954.
Schevitz, R. W., Otwinowski, Z., Joachimiak, A., Lawson, C. L. & Sigler, P. B. (1985). “The three-dimensional structure of trp repressor.” Nature. 317(6040): 782-786.
Schumacher, M. A., Choi, K. Y., Zalkin, H. & Brennan, R. G. (1994) Science 266, 763-770.
Segawa, H., Fukasawa, Y., Miyamoto, K., Takeda, E., Endou, H. & Kanai, Y. (1999) J Biol Chem 274, 19745-19751.
Van der Meer, B. W., Cooker, G. I. & Chen, S. Y. S. (1994) Resonance energy transfer (VCH Publishers, New York).
van Kuppeveld, F. J. M., W. J. G. Melchers, P. H. G. M. Willems, T. W. J. Gadella, (2002). “Homomultimerization of the coxsackievirus 2B protein in living cells visualized by fluorescence resonance energy transfer microscopy Journal of Virology 76(18): 9446-56.
Verrey, F. (2003) Eur J Physiol 44, 529-533.
Yanagida, O., Kanai, Y., Chairoungdua, A., Kim, D. K., Segawa, H., Nii, T., Cha, S. H., Matsuo, H., Fukushima, J., Fukasawa, Y., Tani, Y., Taketani, Y., Uchino, H., Kim, J. Y., Inatomi, J., Okayasu, I., Miyamoto, K., Takeda, E., Goya, T. & Endou, H. (2001) Biochim Biophys Acta 1514, 291-302.
Yanofsky, C. (1981) Nature 289, 751-8.
Zhang, R. G., Joachimiak, A., Lawson, C. L., Schevitz, R. W., Otwinowski, Z. & Sigler, P. B. (1987). “The crystal structure of trp aporepressor at 1.8 A shows how binding tryptophan enhances DNA affinity.” Nature. 327(6123): 591-597.

What is claimed:

1. An isolated nucleic acid which encodes a ligand binding fluorescent indicator, the indicator comprising:
at least one ligand binding protein moiety of a multimeric ligand binding protein complex;
a donor fluorophore moiety fused to the ligand binding protein moiety; and
an acceptor fluorophore moiety fused to the ligand binding protein moiety;
wherein fluorescence resonance energy transfer (FRET) between the donor moiety and the acceptor moiety is altered when the donor moiety is excited and said ligand binds to the ligand binding protein moiety.

2. The isolated nucleic acid of claim 1, wherein said multimeric ligand binding protein complex is selected from the group consisting of dimers, trimers, tetramers and hexamers.

3. The isolated nucleic acid of claim 2, wherein said multimeric ligand binding protein complex is a dimer.

4. The isolated nucleic acid of claim 3, wherein said multimeric ligand binding protein complex is a homodimer.

5. The isolated nucleic acid of claim 1, comprising at least two ligand binding protein moieties from separate proteins of a multimeric ligand binding protein complex.

6. The isolated nucleic acid of claim 5, wherein said multimeric ligand binding protein complex is selected from the group consisting of dimers, trimers, tetramers and hexamers.

7. The isolated nucleic acid of claim 6, wherein said multimeric ligand binding protein complex is a dimer.

8. The isolated nucleic acid of claim 7, wherein said multimeric ligand binding protein complex is a homodimer.

9. The isolated nucleic acid of claim 7, wherein said ligand binding fluorescent indicator comprises a ligand binding single chain dimer fused to donor and acceptor fluorophores.

10. The isolated nucleic acid of claim 5, wherein said ligand binding fluorescent indicator comprises a structure according to the following formula (I):
A-B-C-D, (I)
wherein A and C are fluorophore moieties, and B and D are ligand binding protein moieties.

11. The isolated nucleic acid of claim 5, wherein said ligand binding fluorescent indicator comprises a structure according to the following formula (I):
A-B-C-D, (I)
wherein A and C are ligand binding protein moieties, and B and D are fluorophore moieties.

12. The isolated nucleic acid of claim 5, wherein said ligand binding fluorescent indicator comprises a structure according to the following formula (I):
A-B-C-D, (I)
wherein A and D are ligand binding protein moieties, and B and C are fluorophore moieties.

13. The isolated nucleic acid of claim 5, wherein said ligand binding fluorescent indicator comprises a structure according to the following formula (I):
A-B-C-D, (I)
wherein A and D are fluorophore moieties, and B and C are ligand binding protein moieties.

14. The isolated nucleic acid of claim 1, wherein said multimeric ligand binding protein complex is selected from the group consisting of repressor proteins, enzymes, ligands, nucleic acid binding proteins, growth regulatory factors, differentiative factors, and chemotactic factors, hormone receptors, steroid receptors, serotonin receptors, dopamine receptors, metabotropic and ionotropic glutamate receptors, insulin receptors, IGF1 receptors, G-protein-coupled receptors, immune cell receptors and antibodies.

15. The isolated nucleic acid of claim 14, wherein said multimeric ligand binding protein complex is a bacterial repressor protein.

16. The isolated nucleic acid of claim 15, wherein the bacterial repressor protein is selected from the group consisting of lactose, galactose, purine, tetracycline, tyrosine, multidrug-binding protein QacR, arabinose (AraC), mercury (MerR), and tryptophan repressor proteins, histone deacetylase (HDAC), MEF2-interacting transcription repressor (MITR), silencing mediator for retinoid and thyroid hormone receptors (SMRT), nuclear corepressor (N-CoR), Small Unique Nuclear receptor CoRepressor (SUN-CoR), TG interacting factor (TGIF).

17. The isolated nucleic acid of claim 16, wherein the bacterial repressor protein is a tryptophan repressor protein.

18. The isolated nucleic acid of claim 16, wherein the bacterial repressor protein is a purine repressor protein.

19. The isolated nucleic acid of claim 1, wherein the donor and acceptor moieties are genetically fused to said binding protein moiety.

20. The isolated nucleic acid of claim 19, wherein the donor and acceptor moieties are genetically fused to the termini of the binding protein moiety.

21. The isolated nucleic acid of claim 19, wherein one or both the donor and acceptor moieties are genetically fused to an internal position of said ligand binding protein moiety.

22. The isolated nucleic acid of claim 1, wherein said donor fluorophore is selected from the group consisting of a GFP, a CFP, a BFP, a YFP, a dsRED, CoralHue Midoriishi-Cyan (MiCy) and monomeric CoralHue Kusabira-Orange (mKO).

23. The isolated nucleic acid of claim 1, wherein said acceptor fluorophore moiety is selected from the group consisting of a GFP, a CFP, a BFP, a YFP, a dsRED, CoralHue Midoriishi-Cyan (MiCy) and monomeric CoralHue Kusabira-Orange (mKO).

24. The isolated nucleic acid of claim 22, wherein said donor fluorophore moiety is a genetically altered version of eCFP.

25. The isolated nucleic acid of claim 24, wherein said ligand binding moiety nucleic acid sequence contains the sequence SEQ ID NO: 1.

26. The isolated nucleic acid of claim 1, wherein said acceptor fluorophore moiety is a genetically altered version of YFP VENUS.

27. The isolated nucleic acid of claim 26, wherein said fluorophore nucleic acid sequence is selected from the group consisting of the sequence SEQ ID NOs: 2, 4, and 6.

28. A cell expressing the nucleic acid of claim 1.

29. An expression vector comprising the nucleic acid of claim 1.

30. A cell comprising the vector of claim 29.

31. The expression vector of claim 29 adapted for function in a prokaryotic cell.

32. The expression vector of claim 29 adapted for function in a eukaryotic cell.

33. The cell of claim 30, wherein the cell is a prokaryote.

34. The cell of claim 33, wherein the cell is E. coli.

35. The cell of claim 26, wherein the cell is a eukaryotic cell.

36. The cell of claim 35, wherein the cell is a yeast cell.

37. The cell of claim 35, wherein the cell is an animal cell.

38. The cell of claim 35, wherein said cell is a plant cell.

39. A transgenic animal expressing the nucleic acid of claim 1.

40. A transgenic plant expressing the nucleic acid of claim 1.

41. The isolated nucleic acid of claim 1, further comprising one or more nucleic acid substitutions that modify the affinity of the ligand binding protein moiety to said ligand.

42. A ligand binding fluorescent indicator encoded by the nucleic acid of claim 1.

43. A method of detecting changes in the level of a ligand in a sample, comprising:
(a) providing a cell expressing the nucleic acid of claim 1 and a sample comprising said ligand; and
(b) detecting a change in FRET between said donor fluorophore moiety and said acceptor fluorophore moiety,
wherein a change in FRET between said donor moiety and said acceptor moiety indicates a change in the level of said ligand in the sample.

44. The method of claim 43, wherein the step of determining FRET comprises measuring light emitted from the acceptor fluorophore moiety.

45. The method of claim 43, wherein determining FRET comprises measuring light emitted from the donor fluorophore moiety, measuring light emitted from the acceptor fluorophore moiety, and calculating a ratio of the light emitted from the donor fluorophore moiety and the light emitted from the acceptor fluorophore moiety.

46. The method of claim 43, wherein the step of determining FRET comprises measuring the excited state lifetime of the donor moiety.

47. The method of claim 43, wherein said cell is contained in vivo.

48. The method of claim 43, wherein said cell is contained in vitro.

49. The method of claim 43, wherein fluorescence resonance energy transfer (FRET) between the donor moiety and the acceptor moiety is increased when the donor moiety is excited and said ligand binds to the ligand binding protein moiety.

50. The method of claim 43, wherein fluorescence resonance energy transfer (FRET) between the donor moiety and the acceptor moiety is decreased when the donor moiety is excited and said ligand binds to the ligand binding protein moiety.


SEQUENCE LISTING (16 sequences)

SEQ ID NO: 1 - protein, 108 aa, Escherichia coli
MAQQSPYSAAMAEQRHQEWLRFVDLLKNAYQNDLHLPLLNLMLTPDEREALGTRVRIVEELLRGEMSQRELKNELGAGIATITRGSNSLKAAPVELRQWLEEVLLKSD

SEQ ID NO: 2 - DNA, 4708 bp, Artificial sequence
  DNA sequence of pTK164
atctcgatcccgcgaaattaatacgactcactatagggagaccacaacggtttccctctagataattttgtttaactttaagaaggagatatacatatgcggggttctcatcatcatcatcatcatggtatggctagcatgactggtggacagcaaatgggtcgggatctgtacgacgatgacgataaggatccgggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactgatatcacaagtttgtacaaaaaagctgaacgagcccaacaatcaccctattcagcagcgatggcagaacagcgtcaccaggagtggttacgttttgtcgacctgcttaagaatgcctaccaaaacgatctccatttaccgttgttaaacctgatgctgacgccagatgagcgcgaagcgttggggactcgcgtgcgtattgtcgaagagctgttgcgcggcgaaatgagccagcgtgagttaaaaaatgaactcggcgcaggcatcgcgacgattacgcgtggatctaacagcctgaaagccgcgcccgtcgagctgcgccagtggctggaagaggtgttgctgaaaagcgataacccagctttcttgtacaaagtggtgatatcggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtgatacactattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagagtgacaccacgatgcctgtagcaatgccaacaacgttgcgcaaactattaactggcgaactacttactctagcttcccggcaacaattaatagactgaatggaggcggataaagttgcaggaccacttctgcgctcggcccttccggctggctggtttattgctgataaatctggagccggtgagcgtgggtctcgcggtatcattgcagcactggggccagatggtaagcgctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 3 - protein, 633 aa, Artificial sequence
  Protein sequence of FLIPW-CTY
MRGSHHHHHHGMASMTGGQQMGRDLYDDDDKDPGRMVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITDITSLYKKAERAQQSPYSAAMAEQRHQEWLRFVDLLKNAYQNDLHLPLLNLMLTPDEREALGTRVRIVEELLRGEMSQRELKNELGAGIATITRGSNSLKAAPVELRQWLEEVLLKSDNPAFLYKVVISVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 4 - DNA, 5035 bp, Artificial sequence
  DNA sequence of pTK203
gatccgggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactgatatcacaagtttgtacaaaaaagctgaacgagcccaacaatcaccctattcagcagcgatggcagaacagcgtcaccaggagtggttacgttttgtcgacctgcttaagaatgcctaccaaaacgatctccatttaccgttgttaaacctgatgctgacgccagatgagcgcgaagcgttggggactcgcgtgcgtattgtcgaagagctgttgcgcggcgaaatgagccagcgtgagttaaaaaatgaactcggcgcaggcatcgcgacgattacgcgtggatctaacagcctgaaagccgcgcccgtcgagctgcgccagtggctggaagaggtgttgctgaaaagcgataacccagctttcttgtacaaagtggtgatatcggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtgatacactattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagagtgacaccacgatgcctgtagcaatgccaacaacgttgcgcaaactattaactggcgaactacttactctagcttcccggcaacaattaatagactgaatggaggcggataaagttgcaggaccacttctgcgctcggcccttccggctggctggtttattgctgataaatctggagccggtgagcgtgggtctcgcggtatcattgcagcactggggccagatggtaagcgctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagatctcgatcccgcgaaattaatacgactcactatagggagaccacaacggtttccctctagataattttgtttaactttaagaaggagatatacatatgcggggttctcatcatcatcatcatcatggtatggctagcatgactggtggacagcaaatgggtcgggatctgtacgacgatgacgataaggatccggcccaacaatcaccctattcagcagcgatggcagaacagcgtcaccaggagtggttacgttttgtcgacctgcttaagaatgcctaccaaaacgatctccatttaccgttgttaaacctgatgctgacgccagatgagcgcgaagcgttggggactcgcgtgcgtattgtcgaagagctgttgcgcggcgaaatgagccagcgtgagttaaaaaatgaactcggcgcaggcatcgcgacgattacgcgtggatctaacagcctgaaagccgcgcccgtcgagctgcgccagtggctggaagaggtgttgctgaaaagcgag

SEQ ID NO: 5 - protein, 742 aa, Artificial sequence
  Protein sequence of FLIPW-TCTY
MRGSHHHHHHGMASMTGGQQMGRDLYDDDDKDPAQQSPYSAAMAEQRHQEWLRFVDLLKNAYQNDLHLPLLNLMLTPDEREALGTRVRIVEELLRGEMSQRELKNELGAGIATITRGSNSLKAAPVELRQWLEEVLLKSEDPGRMVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITDITSLYKKAERAQQSPYSAAMAEQRHQEWLRFVDLLKNAYQNDLHLPLLNLMLTPDEREALGTRVRIVEELLRGEMSQRELKNELGAGIATITRGSNSLKAAPVELRQWLEEVLLKSDNPAFLYKVVISVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 6 - DNA, 5038 bp, Artificial sequence
  DNA sequence of pTK204
agcttatggcccaacaatcaccctattcagcagcgatggcagaacagcgtcaccaggagtggttacgttttgtcgacctgcttaagaatgcctaccaaaacgatctccatttaccgttgttaaacctgatgctgacgccagatgagcgcgaagcgttggggactcgcgtgcgtattgtcgaagagctgttgcgcggcgaaatgagccagcgtgagttaaaaaatgaactcggcgcaggcatcgcgacgattacgcgtggatctaacagcctgaaagccgcgcccgtcgagctgcgccagtggctggaagaggtgttgctgaaaagcgattgaaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtgatacactattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagagtgacaccacgatgcctgtagcaatgccaacaacgttgcgcaaactattaactggcgaactacttactctagcttcccggcaacaattaatagactgaatggaggcggataaagttgcaggaccacttctgcgctcggcccttccggctggctggtttattgctgataaatctggagccggtgagcgtgggtctcgcggtatcattgcagcactggggccagatggtaagcgctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagatctcgatcccgcgaaattaatacgactcactatagggagaccacaacggtttccctctagataattttgtttaactttaagaaggagatatacatatgcggggttctcatcatcatcatcatcatggtatggctagcatgactggtggacagcaaatgggtcgggatctgtacgacgatgacgataaggatccgggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactgatatcacaagtttgtacaaaaaagctgaacgagcccaacaatcaccctattcagcagcgatggcagaacagcgtcaccaggagtggttacgttttgtcgacctgcttaagaatgcctaccaaaacgatctccatttaccgttgttaaacctgatgctgacgccagatgagcgcgaagcgttggggactcgcgtgcgtattgtcgaagagctgttgcgcggcgaaatgagccagcgtgagttaaaaaatgaactcggcgcaggcatcgcgacgattacgcgtggatctaacagcctgaaagccgcgcccgtcgagctgcgccagtggctggaagaggtgttgctgaaaagcgataacccagctttcttgtacaaagtggtgatatcggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaaga

SEQ ID NO: 7 - protein, 743 aa, Artificial sequence
  Protein sequence of FLIPW-CTYT
MRGSHHHHHHGMASMTGGQQMGRDLYDDDDKDPGRMVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITDITSLYKKAERAQQSPYSAAMAEQRHQEWLRFVDLLKNAYQNDLHLPLLNLMLTPDEREALGTRVRIVEELLRGEMSQRELKNELGAGIATITRGSNSLKAAPVELRQWLEEVLLKSDNPAFLYKVVISVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKKLMAQQSPYSAAMAEQRHQEWLRFVDLLKNAYQNDLHLPLLNLMLTPDEREALGTRVRIVEELLRGEMSQRELKNELGAGIATITRGSNSLKAAPVELRQWLEEVLLKSD

SEQ ID NO: 8 - DNA, 5065 bp, Artificial sequence
  DNA sequence of pTK205
atctcgatcccgcgaaattaatacgactcactatagggagaccacaacggtttccctctagataattttgtttaactttaagaaggagatatacatatgcggggttctcatcatcatcatcatcatggtatggctagcatgactggtggacagcaaatgggtcgggatctgtacgacgatgacgataaggatccgggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactgatatcacaagtttgtacaaaaaagctgaacgagcccaacaatcaccctattcagcagcgatggcagaacagcgtcaccaggagtggttacgttttgtcgacctgcttaagaatgcctaccaaaacgatctccatttaccgttgttaaacctgatgctgacgccagatgagcgcgaagcgttggggactcgcgtgcgtattgtcgaagagctgttgcgcggcgaaatgagccagcgtgagttaaaaaatgaactcggcgcaggcatcgcgacgattacgcgtggatctaacagcctgaaagccgcgcccgtcgagctgcgccagtggctggaagaggtggtgttgctgaaaagcgaattcggcggcggcggcggcggcggcggcggcatggcccaacaatcaccctattcagcagcgatggcagaacagcgtcaccaggagtggttacgttttgtcgacctgcttaagaatgcctaccaaaacgatctccatttaccgttgttaaacctgatgctgacgccagatgagcgcgaagcgttggggactcgcgtgcgtattgtcgaagagctgttgcgcggcgaaatgagccagcgtgagttaaaaaatgaactcggcgcaggcatcgcgacgattacgcgtggatctaacagcctgaaagccgcgcccgtcgagctgcgccagtggctggaagaggtgttgctgaaaagcgaattcccagctttcttgtacaaagtggtgatatcggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatcgcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtgatacactattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagagtgacaccacgatgcctgtagcaatgccaacaacgttgcgcaaactattaactggcgaactacttactctagcttcccggcaacaattaatagactgaatggaggcggataaagttgcaggaccacttctgcgctcggcccttccggctggctggtttattgctgataaatctggagccggtgagcgtgggtctcgcggtatcattgcagcactggggccagatggtaagcgctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 9 - protein, 750 aa, Artificial sequence
  Protein sequence of FLIPW-CTTY
MRGSHHHHHHGMASMTGGQQMGRDLYDDDDKDPGRMVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITDITSLYKKAERAQQSPYSAAMAEQRHQEWLRFVDLLKNAYQNDLHLPLLNLMLTPDEREALGTRVRIVEELLRGEMSQRELKNELGAGIATITRGSNSLKAAPVELRQWLEEVVLLKSEFGGGGGGGMAQQSPYSAAMAEQRHQEWLRFVDLLKNAYQNDLHLPLLNLMLTPDEREALGTRVRIVEELLRGEMSQRELKNELGAGIATITRGSNSLKAAPVELRQWLEEVLLKSEFPAFLYKVVISVSKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 10 - DNA, 7585 bp, Artificial sequence
  DNA sequence of pTK222
agcttatggcccaacaatcaccctattcagcagcgatggcagaacagcgtcaccaggagtggttacgttttgtcgacctgcttaagaatgcctaccaaaacgatctccatttaccgttgttaaacctgatgctgacgccagatgagcgcgaagcgttggggactcgcgtgcgtattgtcgaagagctgttgcgcggcgaaatgagccagcgtgagttaaaaaatgaactcggcgcaggcatcgcgacgattacgcgtggatctaacagcctgaaagccgcgcccgtcgagctgcgccagtggctggaagaggtgttgctgaaaagcgattgaaagcttgatccggctgctaacaaagcccgaaaggaagctgagttggctgctgccaccgctgagcaataactagcataaccccttggggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactgatatcacaagtttgtacaaaaaagctgaacgagcccaacaatcaccctattcagcagcgatggcagaacagcgtcaccaggagtggttacgttttgtcgacctgcttaagaatgcctaccaaaacgatctccatttaccgttgttaaacctgatgctgacgccagatgagcgcgaagcgttggggactcgcgtgcgtattgtcgaagagctgttgcgcggcgaaatgagccagcgtgagttaaaaaatgaactcggcgcaggcatcgcgacgattacgcgtggatctaacagcctgaaagccgcgcccgtcgagctgcgccagtggctggaagaggtgttgctgaaaagcgataacccagctttcttgtacaaagtggtgatatcggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaaga

SEQ ID NO: 11 - DNA, 54 bp, Artificial sequence
  Forward primer
ggggacaagtttgtacaaaaaagcaggctcggcccaacaatcaccctattcagc

SEQ ID NO: 12 - DNA, 54 bp, Artificial sequence
  Reverse primer
ggggaccactttgtacaagaaagctgggttatcgcttttcagcaacacctcttc

SEQ ID NO: 13 - DNA, 37 bp, Artificial sequence
  Forward primer
ggtaccggaggcggcgttaaccacaccaagtctatcg

SEQ ID NO: 14 - DNA, 36 bp, Artificial sequence
  Reverse primer
ggtaccggcgcctttacgacgatagtcgcggaacgg

SEQ ID NO: 15 - DNA, 46 bp, Artificial sequence
  Primer
gaaatcggcgtcatccccggcccgctggaacagaacaccggcgcag

SEQ ID NO: 16 - DNA, 46 bp, Artificial sequence
  Primer
ctgcgccggtgttctgttccagcgggccggggatgacgccgatttc